(19) United States
(12) Patent Application Publication
(10) Pub. No.: US 2013/0227743 A1
(43) Pub. Date: Aug. 29, 2013

(54) INTEGRATED METHOD FOR HIGH-THROUGHPUT IDENTIFICATION OF NOVEL PESTICIDAL COMPOSITIONS AND USES THEREFOR

(71) Applicants: Christopher J. Grandlic, San Diego, CA (US); Toby H. Richardson, San Diego, CA (US); Janne S. Kerovuo, San Diego, CA (US); Ariel Schwartz, San Diego, CA (US)

(72) Inventors: Christopher J. Grandlic, San Diego, CA (US); Toby H. Richardson, San Diego, CA (US); Janne S. Kerovuo, San Diego, CA (US); Ariel Schwartz, San Diego, CA (US)

(21) Appl. No.: 13/588,621

(22) Filed: Aug. 17, 2012

Related U.S. Application Data
(60) Provisional application No. 61/525,674, filed on Aug. 19, 2011.

(51) Int. Cl. C12N 15/82; C12Q 1/68

(52) U.S. Cl. CPC C12N 15/8286; C12Q 1/6876
USPC 800/279; 435/6.11; 536/24.32; 536/23.71; 435/252.3; 435/419; 800/302; 530/350; 514/4.5; 435/252.31; 435/252.34

(57) ABSTRACT

Methods to rapidly identify nucleic acid sequences encoding novel biotoxins are provided. Particularly, methods to rapidly sample and screen extrachromosomal genetic content of microorganisms for novel sequences of interest are described. Compositions comprising coding sequences for biotoxins, and polypeptides and uses derived therefrom are provided. Compositions and methods are useful, for example, for conferring pesticidal activity to bacteria, plants, plant cells, tissues, and seeds.

### CROSS-REFERENCES TO RELATED APPLICATIONS

This application claims priority under 35 U.S.C. §119(e) to U.S. Provisional Patent Application No. 61/525,674, filed on Aug. 19, 2011, the entire contents of which is herein incorporated by reference.

### INCORPORATION OF SEQUENCE LISTING

The material in the accompanying Sequence Listing is hereby incorporated by reference in its entirety. The accompanying file, named “SGI1530-1_ST25.txt”, was created on Aug. 17, 2012 and is 832 Kb. The file can be accessed using Microsoft Word on a computer that uses Window OS.

### FIELD OF THE INVENTION

This invention relates generally to the field of molecular biology. More specifically, the invention relates to the identification of biotoxin-encoding gene sequences and uses thereof.

### BACKGROUND OF THE INVENTION

Many species of microorganisms, particularly spore-forming gram positive bacterial strains inhabiting soils and other complex ecological communities, produce a wide spectrum of proteinaceous toxins that increase their ability to survive and proliferate. Many of such bacteria often carry extrachromosomal genetic elements including plasmids and episomes that can include a variety of genes. Often, these plasmid-encoded and episome-encoded genes give the strain of a given bacterium important characteristics. For instance, one of the most widely used biocidal pesticides is Crystal (Cry), a protein encoded by extrachromosomal genetic content of subspecies and strains of Bacillus thuringiensis (Bt). To date, a wide variety of Bacillus thuringiensis (Bt) strains and Bt-derived compounds have been used as microbial pesticides. The Bt spores contain crystals, which predominantly comprise one or more Cry and/or Cyt proteins (also known as β-endotoxins), have potent and specific insecticidal activity against various lepidopteran pests. Bt toxins have been used as topical pesticides to protect crops, and more recently the proteins have been expressed in transgenic plants to confer pest resistance. The genes responsible for the production of the insecticidal proteins by these bacterial strains are encoded by extrachromosomal DNA.

While the use of microbial toxins and the genes encoding them in various agricultural applications has become increasingly popular in the past two decades, it remains a cumbersome process to discover and characterize microbial toxin genes with promising potentials for commercial application. Microorganisms represent the largest component of the living world and are widely considered to represent the single largest source of evolutionary and biochemical diversity on the planet. In fact, the total number of microbial cells on Earth is estimated to be at least 10³⁰. Prokaryotes represent the largest proportion of individual organisms, comprising 10⁶ to 10⁸ separate genospecies. In addition, enormous genetic diversity among bacterial extrachromosomal DNA has been reported. Therefore, these microbial genetic materials with tremendous biodiversity remain a largely untapped reservoir of novel genes and compounds with potentials for commercial applications. However, the currently available methods for screening for commercially viable genes from microbes often cannot be applied efficiently to these under-explored resources. For example, the approaches currently used to screen for new crystal toxin proteins of Bacillae species have been largely unchanged since the inception of the field, and primarily relies on time-consuming and rather slow throughput methods. Traditional approaches to identify commercially viable genes and proteins have typically relied on following the function of interest. Typically, new isolates of spore-forming Bacillae are collected from environments, and subsequently subjected to a lengthy multi-step characterization process including (1) microscopic analysis for identification of crystal protein forming strains, (2) nematode and insect feeding and killing assays, (3) degenerative PCR analysis and primer walking to recover full-length toxin gene sequences. A major drawback in such an approach is not only the low throughput, extensive time and effort needed but the fact that discovered gene sequences are determined only after all the effort is already put in.

Newer genomics approaches have attempted to sequence genes as quickly as possible and identify their function by homology to known genes. Efforts to characterize the genomes of microorganisms have been ongoing since tools of molecular biology became available for this purpose. To achieve a much higher sequencing throughput requires technological revolution; therefore numerous commercial companies and scientific labs have come up with many different ways of achieving ultra-high-throughput sequencing. These approaches often involve sequencing and assembling the entire genome of microorganisms, followed by a genome-wide gene annotation before new toxin-encoding sequences can potentially be identified. However, since many of toxin genes reside in the extrachromosomal portion of microbial genome, it remains unclear how efficient it is to sequence entire genomes of a given organism for the purpose of identifying new genetic elements with commercial value. There have been few systematic efforts to characterize genetic materials carried by extrachromosomal DNA of microorganisms, and to use such characterization as a means to rapidly identify microbial genes with commercial applications. One such systematic approach is previously described in U.S. Pat. Appln. No. 20100298207 in which the extrachromosomal DNA content of bacterial strains that could possibly harbor toxin genes of interest was individually extracted, sequenced, assembled, and annotated before toxin genes could be identified. However, further improvements are needed because this approach required that individual microbial strains were isolated and characterized, and the extrachromosomal nucleic acids were isolated from individually cultured strains. In addition, a labor-intensive cloning effort was needed when all DNA libraries were constructed, sequenced and annotated separately and individually for the identification of novel toxin genes in individually processed samples.

Metagenomics is one of today's fastest-developing research areas. The term is derived from the statistical concept of meta-analysis (the process of statistically combining separate analyses) and genomics (the comprehensive analysis of an organism's genetic material). To date, conventional metagenomics is often defined as the application of high-throughput sequencing to DNA obtained directly from environmental samples or series of related samples by bypassing the requirement for obtaining pure cultures for sequencing. To some extent, conventional metagenomics is a derivation of microbial genomics, with the key difference being that it bypasses the requirement for obtaining pure cultures for sequencing. In addition, the samples are obtained from communities rather than isolated populations.

Although metagenomics has been used successfully to identify enzymes with desired activities, it has relied primarily on relatively low-throughput function-based screening or sequence-based screening of environmental DNA clones libraries. Sequence-based metagenomic discovery of complete genes from environmental samples has been limited by microbial species complexity of most environments and the consequent rarity of full-length genes in low-coverage metagenomic assemblies.

Therefore, novel methods are needed to facilitate the rapid and efficient identification of useful nucleotide sequences carried by the extrachromosomal DNA content of microorganisms. Particularly, there is a need to identify more microbial toxin genes with commercial relevance and to do so rapidly and efficiently. One aspect of the present invention provides an integrated screening method as a solution to this long felt need by providing a method to rapidly and efficiently capture the genetic diversity from microorganism genomes and identify novel toxin-encoding sequences of commercial interest, without the need for labor-intensive and relatively low-throughput cloning or sequencing the entire genome of the microorganisms.

### SUMMARY OF THE INVENTION

Methods to rapidly and highly efficient identification of gene sequences encoding biotoxin in microorganisms are described in the present disclosure. Particularly, methods to rapidly sample and screen extrachromosomal genetic content of microorganisms for novel sequences of interest are provided. Isolated nucleic acid molecules encoding novel biotoxins and compositions containing such nucleic acid molecules are also provided in the disclosure. Additionally provided are compositions and methods for conferring pesticidal activity to cells and organisms, for example, microorganisms, plants, plant cells, tissues, and seeds. The nucleic acid sequences and molecules according to the present disclosure can be used in, for examples, making DNA constructs or expression cassettes suitable for transformation and expression in host organisms, including microorganisms and plants. The nucleic acid molecules may also contain synthetic sequences that are designed for optimal expression in a target organism including, but not limited to, a microorganism or a plant. Additionally, polypeptides corresponding to the nucleic acid molecules, methods to produce such polypeptides, and antibodies specifically binding to those polypeptides are also encompassed in the present disclosure.

One aspect of the present invention relates to methods for identifying a nucleic acid sequence encoding a biotoxin. The methods include (a) generating a mixed population of extrachromosomal DNA molecules from a plurality of microbial isolates, (b) establishing a metagenomic sequence dataset comprising nucleic acid sequences derived from said mixed population of extrachromosomal DNA molecules, (c) processing sequence data of said metagenomic sequence dataset to define at least one nucleic acid sequence contig, and (d) identifying a nucleic acid sequence that encodes a biotoxin by comparing said at least one nucleic acid sequence contig from step (c) with known biotoxin sequences.

In some embodiments, the methods according to this aspect of the invention may further include a step of determining the taxonomic classification of the microbial isolates. In some embodiments, the plurality of microbial isolates may be pre-selected for the ability to produce at least one biotoxin. In some preferred embodiments, the methods according to this aspect of the present invention may further include a step of determining whether the nucleic acid sequence identified from step (d) encodes a novel biotoxin. In one embodiment, the nucleic acid sequence of the novel toxin may share less than 30% identity with any known biotoxin sequence. In some embodiments, the nucleic acid sequence of the novel toxin may share less than 60%, or less than 70%, or less than 80%, or less than 90%, or less than 95%, or less than 98%, or less than 99% sequence identity with any known biotoxin sequence. In certain embodiments of the methods according to this aspect, the plurality of microbial isolates includes at least 12 microbial isolates. In some embodiments, the plurality of microbial isolates includes at least 24, or at least 48, or at least 50, or at least 96, or at least 200, or at least 384, or at least 400, or at least 500, or at least 1500 microbial isolates. In a preferred embodiment of this aspect, at least one of the microbial isolates is a bacterium. The bacterium may be, but not limited to, of the following genera Bacillus, Brevibacillus, Clostridia, Paenibacillus, Photorhabdus, Pseudomonas, Serratia, Streptomyces, and Xenorhabdus. In yet other embodiments of this aspect, the metagenomic sequence dataset may be constructed by a direct sequencing procedure that excludes molecular cloning.

Also provided according to another aspect of the present invention are isolated nucleic acid molecules which comprise a nucleic acid sequence that is identified by a method of high-throughput gene identification disclosed herein.

In yet another aspect, the present disclosure provides isolated nucleic acid molecules comprising nucleic acid sequences that hybridize under high stringency conditions to any one of the nucleotide sequences in the Sequence Listing, complements of nucleotide sequences that hybridize under high stringency conditions to any of the nucleotide sequences in the Sequence Listing, and fragments of either; or nucleic acid sequences that exhibit 70% or greater sequence identity to any one of the nucleotide sequences in the Sequence Listing, complements of the nucleotide sequences exhibiting 70% or greater sequence identity to any one of the nucleotide sequences in the Sequence Listing, and fragments of either; or nucleic acid sequences that encode amino acid sequences exhibiting 50% or greater sequence identity to any one of the amino acid sequences in the Sequence Listing.

The disclosure also provides nucleic acid constructs that include the polynucleotides provided herein. The nucleic acid constructs include a heterologous nucleic acid operably linked to a nucleic acid molecule that comprises a nucleic acid sequence corresponding to any one of the nucleotide sequences in the Sequence Listing; or a nucleic acid sequence hybridizing under high stringency conditions to any one of the nucleotide sequences in the Sequence Listing, a complement thereof or a fragment of either; or a nucleic acid sequence exhibiting 70% or greater sequence identity to any one of the nucleotide sequences in the Sequence Listing, a complement thereof or a fragment of either; or a nucleic acid sequence encoding a polypeptide that exhibits 50% or greater sequence identity to any one of the amino acid sequences in the Sequence Listing. In some preferred embodiment, the heterologous nucleic acid is a heterologous promoter. In some other preferred embodiments, the nucleic acid constructs according to this aspect of the present invention are vector constructs. Such vector constructs are useful for transformation and expression of the polynucleotides and polypeptides according to the present invention in transgenic cells and transgenic organisms including, but not limited to, transgenic plants and transgenic microorganisms.

In another aspect, the present disclosure further provides a host cell including a nucleic acid construct that comprises a heterologous nucleic acid operably linked to a nucleic acid molecule that comprises a nucleic acid sequence corresponding to any one of the nucleotide sequences in the Sequence Listing; or a nucleic acid sequence hybridizing under high stringency conditions to any one of the nucleotide sequences in the Sequence Listing, a complement thereof or a fragment of either; or a nucleic acid sequence exhibiting 70% or greater sequence identity to any one of the nucleotide sequences in the Sequence Listing, a complement thereof or a fragment of either; or a nucleic acid sequence encoding a polypeptide that exhibits 50% or greater sequence identity to any one of the amino acid sequences in the Sequence Listing. In some preferred embodiments of this aspect, such host cell may be a plant cell or a microbial cell.

The disclosure also provides host organisms containing host cells that include a nucleic acid construct which comprises a heterologous nucleic acid operably linked to a nucleic acid molecule comprising a nucleic acid sequence corresponding to any one of the nucleotide sequences in the Sequence Listing; or a nucleic acid sequence hybridizing under high stringency conditions to any one of the nucleotide sequences in the Sequence Listing, a complement thereof or a fragment of either; or a nucleic acid sequence exhibiting 70% or greater sequence identity to any one of the nucleotide sequences in the Sequence Listing, a complement thereof or a fragment of either; or a nucleic acid sequence encoding a polypeptide that exhibits 50% or greater sequence identity to any one of the amino acid sequences in the Sequence Listing. In some preferred embodiments of this aspect, such host organism may be a plant or a microorganism. The present disclosure also provides biological samples and progeny derived from the host organisms described above.

In another aspect of the present invention, there is disclosed a method for conferring pesticidal activity to an organism. The method includes introducing into the organism a nucleic acid molecule that includes a nucleic acid sequence corresponding to any one of the nucleotide sequences in the Sequence Listing; or a nucleic acid sequence hybridizing under high stringency conditions to any one of the nucleotide sequences in the Sequence Listing, a complement thereof or a fragment of either; a nucleic acid sequence exhibiting 70% or greater sequence identity to any one of the nucleotide sequences in the Sequence Listing, a complement thereof or a fragment of either; or a nucleic acid sequence encoding a polypeptide that exhibits 50% or greater sequence identity to any one of the amino acid sequences in the Sequence Listing. In a preferred embodiment, the nucleic acid molecule is transcribed and results in an elevated resistance of the organism to a pest as compared to a control organism.

In yet another aspect, the disclosure further provides isolated polypeptides. The isolated polypeptides are encoded by a nucleic acid molecule including a nucleic acid sequence corresponding to any one of the nucleotide sequences in the Sequence Listing; or a nucleic acid sequence hybridizing under high stringency conditions to any one of the nucleotide sequences in the Sequence Listing, a complement thereof or a fragment of either; or a nucleic acid sequence exhibiting 70% or greater sequence identity to any one of the nucleotide sequences in the Sequence Listing, a complement thereof or a fragment of either; or a nucleic acid sequence encoding a polypeptide that exhibits 50% or greater sequence identity to any one of the amino acid sequences in the Sequence Listing. In some preferred embodiments of this aspect, the polypeptides may have a pesticidal activity.

In another aspect of the invention, there are provided compositions comprising a polypeptide encoded by a nucleic acid molecule that comprises a nucleic acid sequence corresponding to any one of the nucleotide sequences in the Sequence Listing; or a nucleic acid sequence hybridizing under high stringency conditions to any one of the nucleotide sequences in the Sequence Listing, a complement thereof or a fragment of either; or a nucleic acid sequence exhibiting 70% or greater sequence identity to any one of the nucleotide sequences in the Sequence Listing, a complement thereof or a fragment of either; or a nucleic acid sequence encoding a polypeptide that exhibits 50% or greater sequence identity to any one of the amino acid sequences in the Sequence Listing. The compositions according to this aspect of the invention may further include one or more of the following features. The polypeptide can be an isolated polypeptide. The polypeptide may have a pesticidal activity. The compositions may further include a carrier. Such carrier may be an agriculturally acceptable carrier. The compositions may additionally comprise an agriculturally effective amount of a pesticidal compound or composition. The additional compound or composition may be an acaricide, a bactericide, a fungicide, an insecticide, a microbicide, a nematicide, a pesticide, or a fertilizer. The compositions may be prepared as a formulation which may be an emulsion, a colloid, a dust, a granule, a pellet, a powder, a spray, or a solution. The compositions may be prepared by centrifugation, concentration, desiccation, extraction, filtration, homogenization, or sedimentation of a culture of microbial cells. In yet other embodiments, the compositions may include from about 1% to about 99% by weight of a polypeptide provided herein.

Also provided in another aspect of the invention is a method for controlling a pest. The method includes contacting or feeding a pest with a pesticidally-effective amount of a polypeptide of the invention as described herein.

In yet another aspect of the invention, provided is a method for producing a polypeptide having pesticidal activity. The method includes culturing a host cell comprising a nucleic acid molecule encoding any one of the polypeptides of the invention as described herein, under conditions in which the nucleic acid molecule is expressed. As such, the polypeptides may be encoded by a nucleic acid molecule that comprises a nucleic acid sequence corresponding to any one of the nucleotide sequences in the Sequence Listing; or a nucleic acid sequence hybridizing under high stringency conditions to any one of the nucleotide sequences in the Sequence Listing, a complement thereof or a fragment of either; or a nucleic acid sequence exhibiting 70% or greater sequence identity to any one of the nucleotide sequences in the Sequence Listing, a complement thereof or a fragment of either; or a nucleic acid sequence encoding a polypeptide that exhibits 50% or greater sequence identity to any one of the amino acid sequences in the Sequence Listing.

Also provided in the present disclosure are purified antibodies that specifically bind to any one of the polypeptides provided herein or a pesticidal fragment thereof. The polypeptides may be encoded by a nucleic acid molecule that comprises a nucleic acid sequence corresponding to any one of the nucleotide sequences in the Sequence Listing; or a nucleic acid sequence hybridizing under high stringency conditions to any one of the nucleotide sequences in the Sequence Listing, a complement thereof or a fragment of either; or a nucleic acid sequence exhibiting 70% or greater sequence identity to any one of the nucleotide sequences in the Sequence Listing, a complement thereof or a fragment of either; or a nucleic acid sequence encoding a polypeptide that exhibits 50% or greater sequence identity to any one of the amino acid sequences in the Sequence Listing.

These and other objects and features of the invention will become more fully apparent from the following detailed description of the invention and the claims.

### DETAILED DESCRIPTION OF THE INVENTION

The present invention relates to compositions and methods useful for modulating pest resistance in organisms, particularly plants or plant cells. Methods to rapidly and efficiently identify gene sequences encoding novel biotoxin are provided. Particularly, methods to rapidly sample and screen extrachromosomal genetic content of microorganisms for novel sequences of interest are described. Isolated nucleic acid molecules encoding novel biotoxins and compositions containing such nucleic acid molecules are also provided in the disclosure. Additionally, compositions and methods for conferring pesticidal activity to bacteria, plants, plant cells, tissues, and seeds are also provided. Additionally, amino acid sequences corresponding to the polynucleotides are encompassed, and antibodies specifically binding to those amino acid sequences are also provided.

Particularly, the nucleic acid molecules of the invention can be used in, for example, the construction of expression vectors for subsequent transformation into organisms of interest, as probes for the isolation of other toxin genes, and for the generation of altered pesticidal proteins by methods known in the art, such as domain swapping or DNA shuffling. The nucleic acid sequences or amino acid sequences may also be synthetic sequences that are designed for optimal expression in a target organism including, but not limited to, a microorganism or a plant. The polypeptides of the invention find use in controlling or killing pest population, particularly lepidopteran, coleopteran, and nematode pest populations, as well as use in the production of compositions with pesticidal activity.

Additionally, microbial cells and plant cells produced using a method in accordance with the present disclosure may be used to produce biomass, microbial products, plant products, e.g., food, feed, biofuel, cosmetic, medicinal, neutraceutical, nutritional, or pharmaceutical products.

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, including mixtures thereof.

Amino acid: As used herein, the term “amino acid” refers to naturally occurring and synthetic amino acids, as well as amino acid analogs and amino acid mimetics that function in a manner similar to the naturally occurring amino acids. Naturally occurring amino acids are those encoded by the genetic code, including D/L optical isomers, as well as those amino acids that are later modified, e.g., hydroxyproline, y-carboxyglutamate, and O-phosphoserine. Amino acid analogs refer to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that functions in a manner similar to a naturally occurring amino acid.

The term “biotoxin” or “toxin”, as used interchangeably herein, is intended to refer to a polypeptide that has toxic activity against one or more pests including, but not limited to, insect pests such as, for example, members of the Lepidoptera, Diptera, and Coleoptera orders, and nematode members of the Nematoda phylum; or a functional homolog of such a polypeptide. The term “biotoxin” is sometimes used to explicitly confirm the biological origin. In some cases, biotoxin proteins are isolated from Bacillus sp. In other embodiments, the toxins can be isolated from other microbial genera, including Clostridium and Paenibacillus. Toxin proteins include amino acid sequences deduced from the full-length nucleotide sequences disclosed herein, and amino acid sequences that are shorter than the full-length sequences, either due to the use of an alternate downstream start site, or due to processing that produces a shorter protein having pesticidal activity. Processing may occur in the organism the protein is expressed in, or in the pest after ingestion of the protein.

Composition: A “composition” is intended to mean a combination of active agent and another compound, carrier or composition, inert (for example, a detectable agent or label or liquid carrier) or active, such as a pesticide.

The terms “control” or “controlling” or grammatical equivalents thereof, as used herein in reference to a pesticidal treatment, are understood to encompass any pesticidal activities or pestistatic (inhibiting, repelling, deterring, preventing, and generally interfering with pest functions to prevent the damage to the host plant) activities of a pesticidal composition against a given pest to effect changes in pest feeding, growth, and/or behavior at any stage of development, including, but not limited to, killing the insect, retarding growth, preventing reproductive capability, and the like. Thus, the terms “control” or “controlling” or grammatical equivalents thereof, not only include killing, but also include such activities as repelling, preventing, deterring, inhibiting or killing egg development or hatching, inhibiting maturation or development, and sterilization of larvae or adult pests.

Control organism: A “control organism” as used in the present invention provides a reference point for measuring changes in phenotype of the subject organism or cell, may be any suitable organism or cell. A control organism or cell may comprise, for example, (a) a wild-type organism or cell, i.e., of the same genotype as the starting material for the genetic alteration which resulted in the subject organism or cell; (b) an organism or cell of the same genotype as the starting material but which has been transformed with a null construct (i.e. a construct which has no known effect on the trait of interest, such as a construct comprising a reporter gene); (c) an organism or cell which is a non-transformed segregant among progeny of a subject organism or cell; (d) an organism or cell which is genetically identical to the subject organism or cell but which is not exposed to the same treatment (e.g., pesticide treatment) as the subject organism or cell; (e) the subject organism or cell itself, under conditions in which the gene of interest is not expressed; or (f) the subject organism or cell itself, under conditions in which it has not been exposed to a particular treatment such as, for example, a pesticide or combination of pesticides and/or other chemicals. In some instances, the term “control organism” refers to an organism or cell used to compare against transgenic or genetically modified organism for the purpose of identifying a modulated phenotype in the transgenic or genetically modified organism. A “control organism” may in some cases refer to an organism that does not contain the exogenous nucleic acid present in the transgenic organism of interest, but otherwise has the same of similar genetic background as such a transgenic organism. In some other instances, an appropriate control organism or cell as used herein may have a different genotype from the subject organism or cell but may share the pesticide-sensitive characteristics of the starting material for the genetic alteration(s) which resulted in the subject organism or cell. For example, a “control plant”, as used for the purpose of this disclosure, refers to a plant cell, seed, plant component, plant tissue, plant organ or whole plant used to compare against transgenic or genetically modified plant for the purpose of identifying a modulated phenotype in the transgenic or genetically modified plant. A “control plant” may in some cases refer to a plant that does not contain the exogenous nucleic acid present in the transgenic plant of interest, but otherwise has the same of similar genetic background as such a transgenic plant. A suitable control plant can be a genetically unaltered or non-transgenic plant of the parental line used to generate a subject transgenic plant. A suitable control plant in some cases can be a non-transgenic segregant from a transformation experiment, or a transgenic plant that contains an exogenous nucleic acid other than the exogenous nucleic acid of interest.

Culturing: The term “culturing”, as used herein, refers to the propagation of a cell or organism on or in media of various kinds such as, for example, liquid, semi-solid or solid medium under suitable conditions wherein the cell or organism can carry out some, if not all, biological processes. For example, a cell that is cultured may be growing or reproducing, and capable of carrying out biological and/or biochemical processes including but not limited to replication, transcription, translation.

Domain: “Domains” are groups of substantially contiguous amino acids in a polypeptide that can be used to characterize protein families and/or parts of proteins. Such domains have a “fingerprint” or “signature” that can comprise conserved primary sequence, secondary structure, and/or three-dimensional conformation. Generally, domains are correlated with specific in vitro and/or in vivo activities. A domain can have a length of from 4 amino acids to 400 amino acids, e.g., 4 to 50 amino acids, or 4 to 20 amino acids, or 4 to 10 amino acids, or 4 to 8 amino acids, or 25 to 100 amino acids, or 35 to 65 amino acids, or 35 to 55 amino acids, or 45 to 60 amino acids, or 200 to 300 amino acids, or 300 to 400 amino acids. As disclosed in greater detail elsewhere herein, conserved regions and conserved domains that are indicative of biotoxin activity have been described extensively in scientific and patent literature.

Effective amount: As used herein, an “effective amount” is an amount sufficient to affect beneficial or desired results. An effective amount can be administered in one or more administrations. In term of pest and/or disease management, treatment, inhibition or protection, an effective amount is that amount sufficient to suppress, stabilize, reverse, slow or delay progression of the target pest infection or disease states. As such, the expression “pesticidally-effective amount” is used herein in reference to that quantity of pesticide treatment which is necessary to obtain a reduction in the level of pest development and/or in the level of pest infection relative to that occurring in an untreated control. For each pesticidal substance or organism, the pesticidally effective amount can be determined empirically for each pest affected in a specific environment. Typically, an effective amount of a given pesticide treatment provides a reduction of at least 20%; or more typically, between 30 to 40%; more typically, between 50-60%; even more typically, between 70 to 80%; and even more typically, between 90 to 95%, relative to the level of pest infection and/or the level of pest development occurring in an untreated control under suitable conditions of treatment. As mentioned above, a pesticidally-effective amount can be administered in one or more administrations.

Exogenous: the “exogenous” when used in reference to a nucleic acid indicates that the nucleic acid is part of a recombinant nucleic acid construct and is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally-occurring sequence by the presence of non-natural sequences linked to the exogenous nucleic acid, e.g., non-native regulatory sequences flanking a native sequence in a recombinant nucleic acid construct. In addition, stably transformed exogenous nucleic acids can be integrated at positions other than the position where the native sequence is found. It will be appreciated that an exogenous nucleic acid may have been introduced into a progenitor, and not into the cell under consideration. For example, a transgenic plant containing an exogenous nucleic acid can be the progeny of a cross between a stably transformed plant and a non-transgenic plant. Such progeny are considered to contain the exogenous nucleic acid.

Expression: As used herein, “expression” refers to the process of converting genetic information of a polynucleotide into RNA through transcription, which is typically catalyzed by an enzyme, RNA polymerase, and into protein, through translation of mRNA on ribosomes.

Functional homolog: The term “functional homolog” as used herein describes those proteins that have at least one characteristic in common. Such characteristics include sequence similarity, biochemical activity, transcriptional pattern similarity and phenotypic activity. Typically, a functional homolog is a polypeptide that has sequence similarity to a reference polypeptide, and that carries out one or more of the biochemical or physiological function(s) of the reference polypeptide. Functional homologs will typically give rise to the same characteristics to a similar, but not necessarily the same, degree. Typically, functionally homologous proteins give the same characteristics where the quantitative measurement due to one of the homologs is at least 20% of the other; more typically, between 30 to 40%; more typically, between 50-60%; even more typically, between 70 to 80%; even more typically, between 90 to 95%; even more typically, between 98 to 100% of the other.

A functional homolog and the reference polypeptide may be naturally occurring polypeptides, and the sequence similarity may be due to convergent or divergent evolutionary events. As such, functional homologs are sometimes designated in the literature as homologs, orthologs, or paralogs. Variants of a naturally-occurring functional homolog, such as polypeptides encoded by mutants or a wild-type coding sequence, may themselves be functional homologs. As used herein, functional homologs can also be created via site-directed mutagenesis of the coding sequence for a biotoxin polypeptide, or by combining domains from the coding sequences for different naturally-occurring biotoxin polypeptides. The term “functional homolog” sometimes applied to the nucleic acid that encodes a functionally homologous polypeptide.

Functional homologs can be identified by analysis of nucleotide and polypeptide sequence alignments. For example, performing a query on a database of nucleotide or polypeptide sequences can identify homologs of biotoxin polypeptides. Sequence analysis can involve BLAST, Reciprocal BLAST, or PSI-BLAST analysis of non-redundant databases using amino acid sequence of an AHAS polypeptide as the reference sequence. Amino acid sequence is, in some instances, deduced from the nucleotide sequence. Typically, those polypeptides in the database that have greater than 40% sequence identity are candidates for further evaluation for suitability as a biotoxin polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains present in biotoxin polypeptides, e.g., conserved functional domains.

Conserved regions can be identified by locating a region within the primary amino acid sequence of a biotoxin polypeptide that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains on the World Wide Web at sanger.ac.uk/Software/Pfam/and pfam.janelia.org/. A description of the information included at the Pfam database is described in, for example, Sonnhammer et al. (Nucl. Acids Res., 26:320-322, 1998), Sonnhammer et al. (Proteins, 28:405-420, 1997); and Bateman et al. (Nucl. Acids Res., 27:260-262, 1999). Conserved regions also can be determined by aligning sequences of the same or related polypeptides from closely related species. Closely related species preferably are from the same family. In some embodiments, alignment of sequences from two different species is adequate. As disclosed in greater detail elsewhere herein, conserved regions and conserved functional domains that are indicative of biotoxin activity have been described extensively in scientific and patent literature.

Typically, polypeptides that exhibit at least about 40% amino acid sequence identity are useful to identify conserved regions. Conserved regions of related polypeptides exhibit at least 45% amino acid sequence identity (e.g., at least 50%, at least 60%, at least 70%, at least 80%, or at least 90% amino acid sequence identity). In some embodiments, a conserved region exhibits at least 92%, 94%, 96%, 98%, or 99% amino acid sequence identity.

Heterologous sequences: the term “heterologous sequences”, as used herein, encompasses heterologous polypeptides and heterologous nucleic acids, and refers to those sequences that are not operatively linked or are not contiguous to each other in nature. For example, a promoter from wheat is considered heterologous to a Bacillus thuringiensis coding region sequence. Also, a promoter from a gene encoding a growth factor from wheat is considered heterologous to a sequence encoding the wheat receptor for the growth factor. Regulatory element sequences, such as UTRs or 3′ end termination sequences that do not originate in nature from the same gene as the coding sequence, are considered heterologous to said coding sequence. Elements operatively linked in nature and contiguous to each other are not heterologous to each other. On the other hand, these same elements remain operatively linked but become heterologous if other filler sequence is placed between them. Thus, the promoter and coding sequences of a wheat gene expressing an amino acid transporter are not heterologous to each other, but the promoter and coding sequence of a wheat gene operatively linked in a novel manner are heterologous.

The term “hybridization”, as used herein, refers generally to the ability of nucleic acid molecules to join via complementary base strand pairing. Nucleic acid molecules or fragment thereof of the present invention are capable of specifically hybridizing to other nucleic acid molecules under certain circumstances. As used herein, two nucleic acid molecules are said to be capable of specifically hybridizing to one another if the two molecules are capable of forming an anti-parallel, double-stranded nucleic acid structure. A nucleic acid molecule is said to be the “complement” of another nucleic acid molecule if they exhibit complete complementarity. As used herein, molecules are said to exhibit “complete complementarity” when every nucleotide of one of the molecules is complementary to a nucleotide of the other. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Conventional stringency conditions are described by Sambrook et al., In: Molecular Cloning, A Laboratory Manual, 2nd Edition, Cold Spring Harbor Press, Cold Spring Harbor, N.Y. (1989), and by Haymes et al. In: Nucleic Acid Hybridization, A Practical Approach, IRL Press, Washington, D.C. (1985). Departures from complete complementarity are therefore permissible, as long as such departures do not completely preclude the capacity of the molecules to form a double-stranded structure. Thus, in order for a nucleic acid molecule or fragment of the present invention to serve as a primer or probe it needs only be sufficiently complementary in sequence to be able to form a stable double-stranded structure under the particular solvent and salt concentrations employed.

Appropriate stringency conditions which promote DNA hybridization include, for example, 6.0× sodium chloride/sodium citrate (SSC) at about 45° C., followed by a wash of 2.0×SSC at about 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed. Information in this regard can be found in Current Protocols in Molecular Biology, John Wiley & Sons, N.Y. (1989), 6.3.1-6.3.6. For example, low stringency conditions may be used to select nucleic acid sequences with lower sequence identities to a target nucleic acid sequence. One may wish to employ conditions such as about 0.15 M to about 0.9 M sodium chloride, at temperatures ranging from about 20° C. to about 55° C. High stringency conditions may be used to select for nucleic acid sequences with higher degrees of identity to the disclosed nucleic acid sequences (Sambrook et al., 1989, supra). High stringency conditions generally involve nucleic acid hybridization in about 2×SSC to about 10×SSC (diluted from a 20×SSC stock solution containing 3 M sodium chloride and 0.3 M sodium citrate, pH 7.0 in distilled water), about 2.5× to about 5×Denhardt's solution (diluted from a 50× stock solution containing 1% (w/v) bovine serum albumin, 1% (w/v) ficoll, and 1% (w/v) polyvinylpyrrolidone in distilled water), about 10 mg/mL to about 100 mg/mL fish sperm DNA, and about 0.02% (w/v) to about 0.1% (w/v) SDS, with an incubation at about 50° C. to about 70° C. for several hours to overnight. Hybridization is typically followed by several wash steps. These wash steps are typically performed by gradually increasing the stringency and comprise 0.5×SSC to about 10×SSC, and 0.01% (w/v) to about 0.5% (w/v) SDS with a 15-min incubation at about 20° C. to about 70° C. Preferably, the nucleic acid segments remain hybridized after washing at least one time in 0.1×SSC at 65° C. In a preferred embodiment, high stringency conditions are provided by pre-hybridization and hybridization at 65° C. in 5×SSC, 5×Denhardt's solution, 100 μg/mL sheared and denatured salmon sperm DNA, and 1% (w/v) SDS for at least three hours, and washing twice with 2×SSC, 0.2% SDS at 65° C.

According to some embodiments of the present application, nucleic acid molecules of the present invention preferably comprise a nucleic acid sequence that hybridizes, under low or high stringency conditions, to any one of the nucleic acid sequences in the Sequence Listing, or any complements thereof, or any fragments of either.

Isolated molecule and substantially purified molecule: an “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. The term “substantially purified”, as used herein, refers to a molecule separated from substantially all other molecules normally associated with it in its native state. More preferably a substantially purified molecule is the predominant species present in a preparation that is, or results, however indirect, from human manipulation of a polynucleotide or polypeptide. A substantially purified molecule may be greater than 60% free, preferably 75% free, more preferably 90% free, and most preferably 95% free from the other molecules (exclusive of solvent) present in the natural mixture. The term “substantially purified” does not encompass molecules present in their native state. For nucleic acids, an “isolated” nucleic acid preferably is free of sequences that naturally flank the nucleic acid (i.e., sequences located at the 5′ and 3′ ends of the nucleic acid) in the cell of the organism from which the nucleic acid is derived. Thus, “isolated nucleic acid” as used herein includes a naturally-occurring nucleic acid, provided one or both of the sequences immediately flanking that nucleic acid in its naturally-occurring genome is removed or absent. Thus, an isolated nucleic acid includes, without limitation, a nucleic acid that exists as a purified molecule or a nucleic acid molecule that is incorporated into a vector or a recombinant organism. A nucleic acid existing among hundreds to millions of other nucleic acids within, for example, cDNA libraries, genomic libraries, or gel slices containing a genomic DNA restriction digest, is not to be considered an isolated nucleic acid. For purposes of the invention, “isolated” when used to refer to nucleic acid molecules also excludes isolated chromosomes. For example, in various embodiments, the isolated toxin encoding nucleic acid molecule can contain less than about 5 kb, 4 kb, 3 kb, 2 kb, 1 kb, 0.5 kb, or 0.1 kb of nucleotide sequences that naturally flank the nucleic acid molecule in the cell from which the nucleic acid is derived. A toxin protein that is substantially free of cellular material includes preparations of protein having less than about 30%, 20%, 10%, or 5% (by dry weight) of non-toxin protein (typically referred to herein as a “contaminating protein”).

The terms “microbial isolate” or “isolated microbial strain”, as used interchangeably herein, refer to a particular species, genus, family, order, or class of microorganism obtained or derived from a sample having more than one microorganism or from a mixed population or microorganisms. As used herein, the term “isolated” as applied to a microorganism (e.g., bacterium or microfungus) refers to a microorganism which has been removed and/or purified from an environment in which it naturally occurs. As such, an “isolated microbial strain” as used herein is a strain that has been removed and/or purified from its natural milieu. Thus, an “isolated” microorganism does not include one residing in an environment in which it naturally occurs. Further, the term “isolated” does not necessarily reflect the extent to which the microbe has been purified. A “substantially pure culture” of the strain of microbe refers to a culture which contains substantially no other microbes than the desired strain or strains of microbe. In other words, a substantially pure culture of a strain of microbe is substantially free of other contaminants, which can include microbial contaminants as well as undesirable chemical contaminants. Further, as used herein, a “biologically pure” strain is intended to mean the strain separated from materials with which it is normally associated in nature. Note that a strain associated with other strains, or with compounds or materials that it is not normally found with in nature, is still defined as “biologically pure.” A monoculture of a particular strain is, of course, “biologically pure.” As used herein, the term “enriched culture” of an isolated microbial strain refers to a microbial culture that contains more than 50%, 60%, 70%, 80%, 90%, or 95% of the isolated strain.

A metagenomic sequence dataset, as used herein, refers to a collection of nucleic acid sequence data that is randomly sampled from and thereby is derived from a plurality of isolated microorganisms. The term metagenomics is derived from the statistical concept of meta-analysis (the process of statistically combining separate analyses) and genomics (the comprehensive analysis of an organism's genetic material).

Nucleic acid and polynucleotide: The terms “nucleic acid” and “polynucleotide” may be used interchangeably herein and refer to both RNA and DNA, including cDNA, genomic DNA, synthetic DNA, and DNA or RNA containing nucleic acid analogs. Polynucleotides can have any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA), transfer RNA, ribosomal RNA, siRNA, micro-RNA, ribozymes, cDNA, DNA/RNA hybrids, recombinant polynucleotides, branched polynucleotides, nucleic acid probes and nucleic acid primers. A polynucleotide may contain unconventional or modified nucleotides.

Operably linked: As used herein, “operably linked” or “operably connected” is intended to mean a functional linkage between two or more sequences. For example, an operable linkage between a polynucleotide of interest and a regulatory sequence (e.g, a promoter) is functional link that allows for expression of the polynucleotide of interest. Operably linked elements may be contiguous or non-contiguous. In this sense, the term “operably linked” refers to the positioning of a regulatory region and a coding sequence to be transcribed in a nucleic acid molecule so that the regulatory region is effective for regulating transcription or translation of the coding sequence of interest. For example, to operably link a coding sequence and a regulatory region, the translation initiation site of the translational reading frame of the coding sequence is typically positioned between one and about fifty nucleotides downstream of the regulatory region. A regulatory region can, however, be positioned as much as about 5,000 nucleotides upstream of the translation initiation site, or about 2,000 nucleotides upstream of the transcription start site. When used to refer to the joining of two protein coding regions, by “operably linked” is intended that the coding regions are in the same translational reading frame. When used to refer to the effect of an enhancer, “operably linked” indicated that the enhancer increases the expression of a particular polypeptide or polynucleotides of interest. Where the polynucleotide or polynucleotides of interest encode a polypeptide, the encoded polypeptide is produced at an elevated level.

Percentage of sequence identity: “percentage of sequence identity” or “percent sequence identity”, as used herein in reference to a nucleic acid sequence or an amino acid sequence, refers to the percentage of identical nucleic acid bases or amino acid residues in a linear sequence of a reference (“query”) molecule as compared to a test (“subject”) molecule when the two sequences are optimally aligned. “Percentage of sequence identity” is determined by comparing two optimally locally aligned sequences over a comparison window defined by the length of the local alignment between the two sequences. The amino acid sequence or nucleic acid sequence in the comparison window may comprise additions or deletions (e.g., gaps or overhangs) as compared to the reference sequence (which does not comprise additions or deletions) for optimal alignment of the two sequences. Local alignment between two sequences only includes segments of each sequence that are deemed to be sufficiently similar according to a criterion that depends on the algorithm used to perform the alignment (e.g. BLAST). The percentage of sequence identity is calculated by determining the number of positions at which the identical nucleic acid base or amino acid residue occurs in both sequences to yield the number of matched positions, dividing the number of matched positions by the total number of positions in the window of comparison and multiplying the result by 100. Optimal alignment of sequences for comparison may be conducted by the local homology algorithm of Smith and Waterman (Add. APL. Math. 2:482, 1981), by the global homology alignment algorithm of Needleman and Wunsch (J. Mol. Biol. 48:443, 1970), by the search for similarity method of Pearson and Lipman (Proc. Natl. Acad. Sci. USA 85: 2444, 1988), by heuristic implementations of these algorithms (NCBI BLAST, WU-BLAST, BLAT, SIM, BLASTZ), or by visual inspection. For purposes of this invention, “percent identity” may also be determined using BLASTX version 2.0 for translated nucleotide sequences and BLASTN version 2.0 for polynucleotide sequences. Given that two sequences have been identified for comparison, GAP and BESTFIT are preferably employed to determine their optimal alignment. Typically, the default values of 5.00 for gap weight and 0.30 for gap weight length are used. The term “substantial sequence identity” between polynucleotide or polypeptide sequences refers to polynucleotide or polypeptide comprising a sequence that has at least 50% sequence identity, preferably at least 70%, preferably at least 80%, more preferably at least 85%, more preferably at least 90%, even more preferably at least 95%, and most preferably at least 96%, 97%, 98% or 99% sequence identity compared to a reference sequence using the programs. In addition, pairwise sequence homology or sequence similarity, as used herein refers to the percentage of residues that are similar between two sequences aligned. Families of amino acid residues having similar side chains have been well defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Query nucleic acid and amino acid sequences can be searched against subject nucleic acid or amino acid sequences residing in public or proprietary databases. Such searches can be done using the National Center for Biotechnology Information Basic Local Alignment Search Tool (NCBI BLAST v 2.18) program. The NCBI BLAST program is available on the interne from the National Center for Biotechnology Information (blast.ncbi.nlm.nih.gov/Blast.cgi). Typically the following parameters for NCBI BLAST can be used: Filter options set to “default”, the Comparison Matrix set to “BLOSUM62”, the Gap Costs set to “Existence: 11, Extension: 1”, the Word Size set to 3, the Expect (E threshold) set to 1e-3, and the minimum length of the local alignment set to 50% of the query sequence length. Sequence identity and similarity may also be determined using GenomeQuest™ software (Gene-IT, Worcester Mass. USA).

Pest: as used herein, the terms “pest” or grammatical equivalents thereof, are understood to refer to undesired organisms that may include, but not limited to, bacteria, fungi, plants (weeds), nematodes, insects, and other pathogenic animals that negatively affect plants and animals by colonizing, attacking, infesting, or infecting them. As such, the term “pesticidal”, as used herein, refers to the ability of a substance or composition to decrease the rate of growth of a pest, i.e., an undesired organism, or to increase the mortality of a pest. The growth rate of pest can be quantified by using any one of a variety of methods known in the art such as, for example, by quantifying the number of viable pests over time.

As used herein, the terms “acaridical”, “aphicidal”, “bactericidal”, “insecticidal”, microbicidal”, or nematicidal”, or grammatical equivalents thereof, are understood to refer to substances or compositions having pesticidal activity against organisms encompassed by the taxonomical classification of root term and also to refer to substances having pesticidal activity against organisms encompassed by colloquial uses of the root term, where those colloquial uses may not strictly follow taxonomical classifications. For example, the term “insecticidal” is understood to refer to substances having pesticidal activity against organisms generally known as insects of the phylum Arthropoda, class Insecta. Further as provided herein, the term is also understood to refer to substances having pesticidal activity against other organisms that are colloquially referred to as “insects” or “bugs” encompassed by the phylum Arthropoda, although the organisms may be classified in a taxonomic class different from the class Insecta. According to this understanding, the term “insecticidal” can be used to refer to substances having activity against arachnids (class Arachnida), in particular mites (subclass Acari/Acarina), in view of the colloquial use of the term “insect.” The term “acaridical” is understood to refer to substances having pesticidal activity against mites (Acari/Acarina) of the phylum Arthropoda, class Arachnida, subclass Acari/Acarina. The term “aphicidal” is understood to refer to substances having pesticidal activity against aphids (Aphididae) of the phylum Arthopoda, class Insecta, family Aphididae. It is understood that all these terms are encompassed by the term “pesticidal” or “pesticide” or grammatical equivalents. It is also understood that these terms are not necessarily mutually exclusive, such that substances known as “insecticides” can have pesticidal activity against organisms of any family of the class Insecta, including aphids, and organisms that are encompassed by other colloquial uses of the term “insect” or “bug” including arachnids and mites. It is understood that “insecticides” can also be known as acaricides if they have pesticidal activity against mites, or aphicides if they have pesticidal activity against aphids.

Promoter: As used herein, a “promoter” is a nucleotide sequence capable of initiating transcription in a cell, such as plant cell or microbial cell, and can drive or facilitate transcription of a nucleotide sequence or fragment thereof of the instant invention. Such promoters need not be of microbial origin or plant origin. For example, promoters derived from plant viruses, such as the CaMV35S promoter or from Agrobacterium tumefaciens, such as the T-DNA promoters, can be useful for the purposes of the present invention. Another non-limiting example is the tac promoter (see, e.g., U.S. Pat. No. 5,840,554) that can be particularly useful for expressing the molecules and sequences in accordance with the present invention in microbial host cells, such as Pseudomonas fluorescens cells.

Polypeptide (may also be used interchangeably with peptide, protein): the term “polypeptide”, as used herein, refers to a compound of two or more subunit amino acids, amino acid analogs, or other peptidomimetics, regardless of post-translational modification, e.g., phosphorylation or glycosylation. The subunits may be linked by peptide bonds or other bonds such as, for example, ester or ether bonds. Full-length polypeptides, truncated polypeptides, point mutants, insertion mutants, splice variants, chimeric proteins, and fragments thereof are encompassed by this definition.

Progeny: As used herein, “progeny” includes descendants of a particular plant or plant line. Progeny of an instant plant include seeds formed on F1, F2, F3, F4, F5, F6 and subsequent generation plants, or seeds formed on BC1, BC2, BC3, and subsequent generation plants, or seeds formed on F1BC1, F1 BC2, F1BC3, and subsequent generation plants. The designation F1 refers to the progeny of a cross between two parents that are genetically distinct. The designations F2, F3, F4, F5 and F6 refer to subsequent generations of self- or sib-pollinated progeny of an F1 plant.

Regulatory region: the term “regulatory region”, as used herein, refers to a nucleotide sequence that influences transcription or translation initiation and rate, and stability and/or mobility of a transcription or translation product in a given host organism. Such regulatory regions can be synthetic or derived from heterologous sources. For example, regulatory regions for use in plants need not be of plant origin. Regulatory sequences include but are not limited to promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof. A regulatory region typically comprises at least a core (basal) promoter. A regulatory region also may include at least one control element, such as an enhancer sequence, an upstream element or an upstream activation region (UAR). For example, a suitable enhancer is a cis-regulatory element (−212 to −154) from the upstream region of the octopine synthase (ocs) gene, which can be useful for driving expression of biotoxin transgenes in plant cells.

Transgenic organism: as used herein, a “transgenic organism” or “recombinant organism” refers to an organism which comprises within its genome a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to include any cell, cell line, callus, tissue, the genotype of which has been altered by the presence of heterologous nucleic acid. The term transgenic includes those transgenics initially so altered as well as those created by sexual crosses or asexual propagation from the initial transgenic. The term transgenic as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutations.

Variant: when referring to polypeptides and nucleic acids, the term “variant” is used herein to denote a polypeptide, protein or polynucleotide molecule with some differences, generated synthetically or naturally, in their base or amino acid sequences as compared to a reference polypeptide or polynucleotide, respectively. For example, these differences include substitutions, insertions, deletions or any desired combinations of such changes in a reference polypeptide or polynucleotide. Polypeptide and protein variants can further consist of changes in charge and/or post-translational modifications (such as glycosylation, methylation, phosphorylation, etc.) “Functional variants” of the regulatory polynucleotide sequences are also encompassed by the compositions of the present invention. Functional variants include, for example, the native regulatory polynucleotide sequences of the invention having one or more nucleotide substitutions, deletions or insertions and which can drive expression of an operably-linked polynucleotide sequence under conditions similar to those under which the native promoter is active. Functional variants of the invention may be created by site-directed mutagenesis, induced mutation, or may occur as allelic variants (polymorphisms). When the term “variant” is used in reference to a microorganism, it typically refers to a microbial strain having identifying characteristics of the species to which it belongs, while having at least one nucleotide sequence variation or identifiably different trait with respect to the parental strain, where the trait is genetically based (heritable).

Vector: the term “vector” refers to a nucleic acid construct designed for transfer between different host cells. As used herein, “vector” refers to a replicon, such as a plasmid, phage, or cosmid, into which another DNA segment may be inserted so as to bring about the replication of the inserted segment. Generally, a vector is capable of replication when associated with the proper control elements. As such, the term “vector” includes cloning vectors and expression vectors, as well as viral vectors and integrating vectors. In particular, an “expression vector” is a vector that includes a regulatory region, thereby capable of expressing DNA sequences and fragments in a host cell (in vivo) and/or in a cell-free environment (in vivo).

All publications and patent applications mentioned in this specification are herein incorporated by reference to the same extent as if each individual publication or patent application was specifically and individually indicated to be incorporated by reference.

No admission is made that any reference constitutes prior art. The discussion of the references states what their authors assert, and the applicants reserve the right to challenge the accuracy and pertinence of the cited documents. It will be clearly understood that although a number of prior art publications are referred to herein, this reference does not constitute an admission that any of these documents forms part of the common general knowledge in the art.

The discussion of the general methods given herein is intended for illustrative purposes only. Other alternative methods and embodiments will be apparent to those of skill in the art upon review of this disclosure.

### Extrachromosomal Genetic Content and Biotoxins

Much of the diversity present in bacterial populations is present on extrachromosomal DNA content, including plasmids and episomes. Strain variation due to plasmid content is well known for Bacillus strains, particularly Bacillus thuringiensis (“Bt”). Insecticidal proteins, such as the Bacillus thuringiensis delta-endotoxin genes which are found predominately on large extrachromosomal DNA molecules, can be rapidly discovered by using the method(s) of the present invention. Furthermore, many Clostridia strains are also known to have large extrachromosomal plasmids, and some of these are known to contain virulence factors, as well as toxins such as iota toxin (see, e.g., Perelle et al., Infect. Immun., 61:5147-5156, 1993; and the references cited therein). In addition, it has been shown that the majority of variability for Clostridia strains appears to occur due to plasmid content (see, e.g., Katayam et al., Mol. Gen. Genet. 250:17-28, 1996). Thus, decoding of the extrachromosomal DNA content of multiple Clostridia strains will quickly capture a large amount of genetic diversity. In addition, there has been report of a homolog of delta-endotoxin gene present in Clostridia sp. (Barloy et al., J. Bacterial. 178:3099-3105, 1996).

Many microbial plasmids are also known to contain virulence factors, important for infectivity or severity of infection by bacterial pathogens. Accordingly, it is likely that many of the proteins expressed by plasmid genomes are likely to have value as vaccines. For example, both plasmids pXO1 and pXO2 of Bacillus anthracis have been reported to encode proteins required for pathogenesis during anthrax infection. pXO2 encodes proteins that produce a protective capsule around the bacterium. The pXO1 plasmid encodes the three proteins of the anthrax toxin complex, lethal factor (LF), edema factor (EF), and the protective antigen (PA). The PA protein (protective antigen) forms the basis of a vaccine for anthrax. The quick and efficient decoding of bacterial plasmids will yield information with which one can create a database of proteins that might serve as effective vaccines.

Tumor-inducing and symbiotic plasmids are common in Agrobacterium and Rhizobium strains (Van Larebeke et al., Nature, 252:169-170, 1974). Thus decoding of bacterial plasmids, especially those from known plant pathogens, is likely to identify genes involved in plant-pathogen interactions including genes involved in or required for both virulence and avirulence.

In a non-limiting exemplification, insecticidal proteins such as the Bacillus thuringiensis delta-endotoxin genes are often found on large extrachromosomal DNA molecules. Thus isolation and sequencing of extrachromosomal DNA from Bacillus strains, such as Bacillus thuringiensis strains is likely to lead to identification of novel delta-endotoxin genes. Such toxin genes are potentially valuable for the control of insect pests.

Bacillus thuringiensis is a Gram-positive spore forming soil bacterium characterized by its ability to produce crystalline inclusions that are specifically toxic to certain orders and species of insects, but are harmless to plants and many other non-targeted organisms. Conventional submerged fermentation techniques can be used to produce Bt spores on a large scale makes Bt bacteria commercially attractive as a source of insecticidal compositions. Compositions including Bacillus thuringiensis strains or their insecticidal proteins are widely used as environmentally-acceptable insecticides to control agricultural insect pests or insect vectors for a variety of human or animal diseases.

Crystal (Cry) proteins (delta-endotoxins) from Bacillus thuringiensis have potent insecticidal activity against predominantly Lepidopteran, Dipteran, and Coleopteran larvae. These proteins also have been reported to show pesticidal activity against Hymenoptera, Homoptera, Phthiraptera, Mallophaga, and Acari pest orders, as well as other invertebrate orders such as Nemathelminthes, Platyhelminthes, and Sarcomastigorphora. There are currently over 600 known species of crystal proteins with a wide range of specificities and toxicities. These crystal proteins and corresponding genes were originally classified primarily based on their structure and insecticidal spectrum (see, e.g., Feitelson, In Advanced Engineered Pesticides, Ed. Kim, L., Marcel Dekker, Inc., New York, N.Y., pp. 63-71, 1993). The major classes were Lepidoptera-specific (I), Lepidoptera- and Diptera-specific (II), Coleoptera-specific (III), Diptera-specific (IV), and nematode-specific (V) and (VI). The proteins were further classified into subfamilies; more highly related proteins within each family were assigned divisional letters such as Cry1A, Cry1B, Cry1C, etc. Even more closely related proteins within each division were given names such as Cry1C1, Cry1C2.

A more recent nomenclature was described for the Cry genes based upon amino acid sequence identity rather than insect target specificity (Crickmore et al., Microbiol. and Mol. Bio. Reviews, 62:807-813, 1998). In this classification, each toxin is assigned a unique name incorporating a primary rank (an Arabic number), a secondary rank (an uppercase letter), a tertiary rank (a lowercase letter), and a quaternary rank (another Arabic number). In the new classification, Roman numerals have been exchanged for Arabic numerals in the primary rank. Proteins with less than 45% sequence identity have different primary ranks, and the criteria for secondary and tertiary ranks are 78% and 95%, respectively.

The crystal protein typically does not exhibit insecticidal activity until it has been ingested and solubilized in the insect midgut. The ingested protoxin is hydrolyzed by proteases in the insect digestive tract to an active toxic molecule. This toxin binds to apical brush border receptors in the midgut of the target larvae and inserts into the apical membrane creating ion channels or pores, resulting in larval death.

Delta-endotoxins generally have five conserved sequence domains, and three conserved structural domains (see, for example, de Maagd et al., Trends Genetics 17:193-199, 2001). The first conserved structural domain consists of seven alpha helices and is involved in membrane insertion and pore formation. Domain II consists of three beta-sheets arranged in a Greek key configuration, and domain III consists of two antiparallel beta-sheets in “jelly-roll” formation (de Maagd et al., 2001, supra). Domains II and III are involved in receptor recognition and binding, and are therefore considered determinants of toxin specificity.

Aside from delta-endotoxins, there are several other known classes of insecticidal and pesticidal protein toxins. Other kinds of insecticidal proteins have been described in B. thuringiensis and Bacillus cereus, among which are the vegetative insecticidal proteins or Vip proteins. The Vip proteins are secreted during vegetative growth and do not exhibit any similarity with the Cry or Cyt toxins. Currently, all Vip-related sequences that have been described fall into three different families, Vip1, Vip2, and Vip3. A classification of these proteins into three classes, seven subclasses, and further subdivisions was recently proposed by the Bacillus thuringiensis nomenclature committee (Crickmore et al., 2005, at www.lifesci.sussex.ac.uk/Home/Neil_Crickmore/Bt/). Vip3 proteins have a different host range, which includes several major lepidopteran pests. Like Cry toxins, Vip3A proteins must be activated by proteases prior to recognition at the surface of the midgut epithelium of specific 80-kDa and 100-kDa membrane proteins different from those recognized by Cry toxins. Apoptosis was initially suggested as a mode of action, but it was recently shown that like Cry toxins, activated Vip3A toxins are pore-forming proteins capable of making stable ion channels in the membrane. The Vip1 and Vip2 proteins are the two components of a binary toxin that exhibits toxicity to coleopterans. Vip1Aa1 and Vip2Aa1 are generally very active against corn rootworms, particularly Diabrotica virgifera virgifera and Diabrotica longicornis. The VIP1NIP2 toxins, together with other binary (“A/B”) toxins. A/B toxins such as VIP, C2, CDT, CST, or the B. anthracis edema and lethal toxins exhibit strong activity on insects by a mechanism believed to involve receptor-mediated endocytosis followed by cellular toxification.

### Description of the Screening Method

The present disclosure provides an integrated approach to rapidly and efficiently identify and isolate useful genes. One aspect of the present invention provides a method to rapid and highly efficient identification of gene sequences encoding biotoxin in microorganisms. Particularly, the method allows for a rapid and efficient sampling and screening of extrachromosomal genetic content of microorganisms for novel sequences of interest. The method involves rapid sequencing and characterization of mixed populations of extrachromosomal DNA molecules derived from a collection of microbial isolates. The method targets extrachromosomal DNA and avoids repeated cloning and sequencing the host chromosomes, thus allowing one to focus on genes that are encoded by extrachromosomal DNA, e.g. biotoxins. The method involves establishing a metagenomic dataset comprising nucleotide sequences deriving from the mixed population of extrachromosomal DNA molecules, processing and comparing the annotated sequences of the metagenomic dataset against known sequences to identify novel nucleotide sequences. In some preferred aspects of the present invention, the processed DNA sequences can be translated in all six frames and the resulting amino acid sequences can be compared against known protein sequences. Microorganisms of particular interest include, but not limited to bacteria, fungi, algae, and the like.

The integrated screening methods described herein can be used to rapidly identify and clone novel genes that have homology to existing genes. Particularly, the screening methods above can be useful for the identification of novel genes that have little homology with known genes, which would be difficult to identify by other methods, such as hybridization.

The workflow of a typical screen begins with the generation of a collection of isolated microbes and proceeds with isolation of extrachromosomal DNA, high throughput sequencing, sequence read processing and assembly. During process of sequence data mining and analysis, genes are called on sequence reads, or sequence contigs, or both. Community composition analysis (i.e. metagenomic data analysis) is employed at several stages of this workflow, and databases are typically needed to facilitate the analysis. All of the steps of this workflow will be described in detail below and throughout the present disclosure.

Environmental samples, including soil, plant tissues, insects and water samples may be collected from diverse ecosystems that harbor native plants with phylogenetic similarity to target crops. Culture-based isolations of plant-associated microbes may be conducted in a multi-phase approach by targeting populations residing in the soil, rhizosphere and phyllosphere. Individual samples may be processed separately or, alternatively, multiple samples from a geographically unique sampling location were pooled together prior further processing, which can be particularly useful in capturing the microbial diversity within an entire region using a single isolation event. Microbial cell extraction methods can be performed on samples, followed by serial dilution and plating onto a highly selective chromogenic medium developed to isolate Bacillus thuringiensis (Bt). Bt isolates can be colony-picked, archived, and grown individually in small-volume cultures in preparation for subsequent extractions of extrachromosomal DNA. Populations of non-Bt microbes can be targeted in a similar manner by plating environmental samples onto various enrichment and isolation media or by selecting specific strains, based on their phylogeny, from existing archives to create composite cultures that may typically be composed of several hundred individual isolate cultures. Large construct plasmid extraction kits (QIAGEN, Inc.) can then be used to isolate extrachromosomal DNA from the composite cultures. In some embodiments of the present invention, modifications may be made to the QIAGEN® recommended workflow to make the lysis procedure more rigorous for lysing Gram-positive cells. The resulting purified extrachromosomal DNA, with minimal genomic contamination, can then be quantified and prepared for next-generation high throughput sequencing.

Since extractions of extrachromosomal DNA can typically be performed on composite cultures, the resulting purified DNA samples are mixed populations of extrachromosomal DNA that are typically derived from hundreds of individual isolates.

After isolation, the pool of extrachromosomal nucleic acids can be subjected to a high-throughput sequencing process to generate metagenomic datasets. Processing step of metagenomic sequence data, which includes assembly, gene prediction and annotation, can be used to identify genes having potential activity of interest. As described in detail below, several toxin genes have been identified by using the method of the present invention, that belong to many major classes of Bt toxins including Cry, VIP and Cyt genes. As reported in Table 1 and set forth in the Sequence Listing, several full-length and partial novel biotoxin-encoding genes were discovered along with many genes already previously discovered.

### Establishing a Metagenomic Sequence Dataset

Metagenomics is currently one of the fastest-developing research areas. The term is derived from the statistical concept of meta-analysis (the process of statistically combining separate analyses) and genomics (the comprehensive analysis of an organism's genetic material). To date, conventional metagenomics is often defined as the application of high-throughput sequencing to DNA obtained directly from environmental samples or series of related samples. To some extent, conventional metagenomics is a derivation of microbial genomics, with the key difference being that it bypasses the requirement for obtaining pure cultures for sequencing. In addition, the samples are obtained from communities rather than isolated populations. In principle, the metagenomic analysis of environmental microbial communities can be divided into two main approaches: function-based and sequence-based screening of metagenomic libraries. Both screening techniques include the isolation of environmental DNA and the construction of small-insert or large-insert libraries (see, e.g. Simon and Daniel, Appl. Environ. Microbiol. 77:1153-1161, 2011).

Although metagenomics has been used successfully to identify enzymes with desired activities, it has relied primarily on relatively low-throughput function-based screening or sequence-based screening of environmental DNA clones libraries. Sequence-based metagenomic discovery of complete genes from environmental samples has been limited by microbial species complexity of most environments and the consequent rarity of full-length genes in low-coverage metagenomic assemblies. The integrated screening method according to one aspect of the present invention provides a solution to this long felt need by providing a method to rapidly and efficiently capture the genetic diversity from microorganism genomes and identify novel sequences of commercial interest, without the need for labor-intensive construction of clone libraries, or sequencing the entire genome of the microorganisms.

Some embodiments of the present invention involve establishing a metagenomic sequence dataset. As discussed above, conventional metagenomics is often defined as the application of high-throughput sequencing to DNA obtained directly from environmental samples or series of related samples by bypassing the requirement for obtaining pure cultures for sequencing. For the purpose of this application, the term “metagenomic sequence data” refers to randomly sampled DNA sequence data that is derived from a plurality of isolated microbes. Sequence data from metagenomic sequence datasets are often assembled into larger contigs. In general, the term “contig” (from “contiguous”) refers to a set of overlapping nucleic acid sequences that together represent a consensus region of a nucleic acid molecule. In a typical genome sequencing projects, a contig refers to overlapping sequence data (reads), resulting from the reassembly of the small DNA fragments generated by bottom-up sequencing strategies, which involves shearing genomic DNA into many small fragments (“bottom”), sequencing these fragments, reassembling them back into contigs and eventually the entire genome (“up”). As such, the term “contig” as used herein refers to contiguous extrachromosomal DNA stretches comprising a plurality of overlapping reads. A metagenomic dataset typically comprises at least 10 Mbp, at least 20 Mbp, preferably at least 30 Mbp, more preferably at least 40 Mpb, and most preferably at least 50 Mbp of short sequence reads data, that can subsequently be used for in silico sequence mining for genes and sequences of commercial interest, such as biotoxin-encoding genes.

### Sequencing Technologies Suitable for Practicing the Method of the Invention

The sequence of extrachromosomal nucleic acid molecules may be determined by using a variety of techniques, particularly the next-generation high-throughput sequencing technologies, which are sometimes referred to as massively parallel sequencing techniques. These high-throughput sequencing techniques are well known and described in the technical and scientific literature, for example, in a review by Lin et al. (Recent patents on Biomedical Engineering, 1:60-67, 2008) and the references cited therein.

In some embodiments, sequencing may be performed directly on the extrachromosomal nucleic acid molecules by using direct sequencing procedures that do not require molecular cloning. Although the cloning of the nucleic acid molecules is relatively straightforward, direct sequencing of nucleic acids typically eliminates the need in subcloning and production of many shotgun libraries, minimizes the number of sequencing reactions, and dramatically accelerates the acquisition of sequence information and the assembly of complete sequences. The advantages of direct nucleic acid sequencing include elimination of cloning artifacts and cross-contamination of libraries or PCR reactions. This is extremely important for production sequencing of closely related organisms, as it provides non-biased complete coverage of the genomes with low number of redundant sequencing reactions and results in significant savings on data processing. Common techniques for direct sequencing of nucleic acids are known in the art. See, e.g., Lin et al. (2008, supra); Lilian et al., (Quarterly Rev. Biophysics, 169-200, 2002).

Sequencing of the extrachromosomal nucleic acid molecules may also be performed by one of several conventional sequencing methods including, but not limited to, conventional gel-based technologies as well as those that encompass sequencing by synthesis (SBS), sequencing by ligation, sequencing by hybridization, and many more recent sequencing technologies using nano-transistor array, scanning tunneling microscopy and nanowire molecule sensors, etc.

Common gel-based technologies are essential derived from the methodology developed by Sanger et al. in 1970's (Sanger et al., 1977), which involves sequencing by chain termination and gel separation. In such method, a mixed population of nucleic acid fragments representing terminations at each base was generated using ‘terminator’—the 2′,3′-dideoxy and arabinonucleoside analogues of the normal deoxynucleoside triphosphates. They are run on an electrophoretic gel and the sequence can be ‘read’ from the order of fragments in the gel. A similar sequencing method that relies on chemical degradation of nucleic acid fragments at each base was also developed by Maxam and Gilbert (Proc. Natl. Acad. Sci. USA, 1977).

Sequencing by synthesis using fluorophore-labeled, reversible-terminator nucleotides is the most common platform of sequencing by synthesis. It is sometimes named “fluorescent in situ sequencing” (FISSEQ). It usually involves these following steps: attaching the DNA to be sequenced in a solid surface, then adding polymerase and labeled nucleotides with cleavable chemical group to cap an-OH group at a 3′-position of the deoxyribose so that incorporation of the nucleotides terminates the reaction. The sequence can be read from the labels used for nucleotides. The Pyrosequencing technology is another SBS technology developed by Ronaghi et al. (Ronaghi et al., Anal. Biochem. 242, 1: 84-9, 1996; Ronaghi, Genome Res. 11:3-11, 2001). In brief, it is based on the detection of pyrophosphate (PPi) released during DNA synthesis when inorganic PPi is released after nucleotide incorporation by DNA polymerase. The released PPi is then converted to ATP by ATP sulfurylase. A luciferase reporter enzyme uses the ATP to generate light, which is then detected by a charge coupled device (CCD) camera. The light signal is proportional to the number of nucleotides incorporated (e.g. A, TT, CCC etc) and because the G, A, T, and C nucleotides are added stepwise in a sequencing cycle, the DNA sequences are easily derived. Pyrosequencing has evolved into an ultra-high throughput sequencing technology with the combination of several technologies such as template carrying microbeads deposited in microfabricated picoliter-sized reaction wells connecting to optical fibers. Several commercial sequencing platforms based on the pyrosequencing technology are currently available, such as Genome Sequencer 20 System and the Genome Sequencer FLX System from 454 Life Science/Roche Diagnostics, and the “Fluorescent Resonance Energy Transfer (FRET)” technology commercialized by Visigen Biotechnolgies Inc (see, e.g., U.S. Pat. Appln. Nos. US20070172869, US20070172860, and US200701728190. Other SBS-based technologies including, but not limited to, those marketed by Intelligent Bio-Systems Inc. (see, e.g., European Pat. Appln. No. EP1790736), Affymetrix Inc. (see, e.g., U.S. Pat. Appln. No. US20070105131) may also be useful. In some embodiments of the invention, the Genome Analyzer™ system (e.g., U.S. Pat. Appln. No. US20077232656), which is also based on an SBS technology, from Illumina Inc. is particularly preferred.

One skilled in the art will recognize that it is advantageous and often necessary to generate sequence data from both ends (as known as pair-end, dual-end or double ended sequencing) of a template DNA fragment to confirm or help shotgun sequence assemblies. Pair-end sequencing will also be useful for characterization of genomic rearrangement and insertion and deletion, such as in cancer genome characterization. A variety of “double-end sequencing” technologies are well known, such as those described in U.S. Pat. Appln. Nos. US20077244567, US20060024681, US20070172839, US20060292611, US20077270951, and US20077282337, and may be used for the method of the present invention.

In certain embodiments, other high-throughput sequencing methods based on polony amplification and FISSEQ may be used. In brief, polony amplification is a method to amplify DNAs in situ on a thin polyacrylamide film. The DNA movement is limited in the polyacrylamide gel, so the amplified DNAs are localized in the gel and form the so-called “polonies”, polymerase colonies. Up to 5 million polonies (i.e. 5 million PCRs) can form on a single glass microscope slide. Variants of the polony sequencing method, which include polonyfluorescent-in situ-sequencing beads and PMAGE (for “polony multiplex analysis of gene expression”, which combines polony amplification and a sequence-by-ligation method), may also be used for the method of the invention.

Other high-throughput sequencing technologies, devices and systems that may also be used to practice the present invention include those that encompass nanopore sequencing (see, e.g., U.S. Pat. Appln. Nos. US20070190542, US20070042366, US20070048745, US20060231419, and US20070178507), and sequencing by hybridization (SBH) (see, e.g., U.S. Pat. Appln. No. US20070178516, US20077276338, and US20060287833).

A variety of high-throughput sequencing by ligation technologies may also be used for the method of the present invention. Examples of such technologies include, but not limited to, the “Massively Parallel Signature Sequencing” technology (see, e.g. Brenner et al., Proc. Natl. Acad. Sci. USA, 2000; U.S. Pat. Appln. No. US20006013445). In some versions of this technology, DNA molecules are amplified in parallel onto microbeads by emulsion polymerase chain reaction. Millions of beads are then immobilized in a polyacrylamide gel and sequenced using sequencing by ligation method. Devices and systems commercialized by Applied Biosystems/Life Inc. such as SOLiD (Supported Oligo Ligation Detection) may be particularly useful. A more recent version, which is based on a similar sequence-by-ligation method in combination with emulsion PCR, may also be used.

One skilled in the art will recognize that it is advantageous and often necessary to deploy combinations of different sequencing technologies for producing better-quality assembly and annotation of microbial metagenomic sequence data.

### Methods for Taxonomic Identification

Once a microorganism has been selected by the screening methods disclosed by the present invention, it is often beneficial to identify them taxonomically. One of skill in the art will appreciate that the taxonomic classification of microorganism isolates can be determined by a variety of techniques, including but not limited to (1) hybridization of a nucleic acid probe to a nucleic acid molecule of said microbial isolates; (2) amplification of a nucleic acid molecule of said microbial isolates; (3) immuno-detection of a molecule of said microbial isolates; (4) sequencing of a nucleic acid molecule derived from said microbial isolates; or a combination of two or more of these techniques.

Organism identification can therefore involve up to several different levels of analysis, and each analysis can be based on a different characteristic of the organism. Such analyses can include nucleic acid-based analysis (e.g., analysis of individual specific genes, either as to their presence or their exact sequence, or expression of a particular gene or a family of genes), protein-based analysis (e.g., at a functional level using direct or indirect enzyme assays, or at a structural level using immuno-detection techniques), and so forth.

Prior to carrying out intensive molecular analysis of isolated cultures, it may be useful to confirm that the microbial culture arose from a single cell, and is therefore a pure culture (except where, as discussed elsewhere in this disclosure, microorganisms are intentionally mixed). Microorganisms can often be distinguished based on direct microscopic analysis (do all of the cells in a sample look the same on examination), staining characteristics, simple molecular analysis (such as a simply restriction fragment length polymorphism (RFLP) determination), and so forth. In certain embodiments of the invention, however, it is not absolutely necessary to perform this purity confirmation step, as mixed microbial cultures will be apparent in subsequent analysis.

a. Nucleic Acid-Based Analysis:

In certain embodiments of the invention, methods provided for identifying microorganisms include amplifying and sequencing genes from very small numbers of cells. The provided procedures therefore overcome the problems of concentrating cells and their DNA from dilute suspensions. The provided procedures can be used to identify cells by gene sequence or to identify cells that have particular genes or gene families.

The term “nucleic acid amplification” generally refers to techniques that increase the number of copies of a nucleic acid molecule in a sample or specimen. Techniques useful for nucleic acid amplification are well known in the art. An example of nucleic acid amplification is the polymerase chain reaction (PCR), in which a biological sample collected from a subject is contacted with a pair of oligonucleotide primers, under conditions that allow for the hybridization of the primers to nucleic acid template in the sample. The primers are extended under suitable conditions, dissociated from the template, and then re-annealed, extended, and dissociated to amplify the number of copies of the nucleic acid. Other examples of in vitro amplification techniques include strand displacement amplification; transcription-free isothermal amplification; repair chain reaction amplification; ligase chain reaction; gap filling ligase chain reaction amplification; coupled ligase detection and PCR; and RNA transcription-free amplification.

In addition to the illustrative example primers provided herein, primers have also been designed, and new ones are continually being designed, for individual species or phylogenetic groups of microorganisms. Such narrowly targeted primers can be used with the methods described herein to screen and/or identify specifically only the microorganisms of interest.

Methods for preparing and using nucleic acid primers are described, for example, in Sambrook et al. (In Molecular Cloning: A Laboratory Manual, CSHL, New York, 1989), Ausubel et al. (ed.) (In Current Protocols in Molecular Biology, John Wiley & Sons, New York, 1998). Amplification primer pairs can be derived from a known sequence, for example, by using computer programs intended for that purpose such as Primer (Whitehead Institute for Biomedical Research, Cambridge, Mass.). One of ordinary skill in the art will appreciate that the specificity of a particular probe or primer increases with its length. Thus, for example, a primer comprising 30 consecutive nucleotides of an rRNA-encoding nucleotide or flanking region thereof will anneal to a target sequence with a higher specificity than a corresponding primer of only 15 nucleotides. Thus, in order to obtain greater specificity, probes and primers can be selected that comprise at least 20, 25, 30, 35, 40, 45, 50 or more consecutive nucleotides of a target nucleotide sequence such as the 16S rRNA.

Common techniques for the preparation of nucleic acids useful for nucleic acid applications (e.g., PCR) include phenol/chloroform extraction or use of one of the many DNA extraction kits that are available on the market. Another way that DNA can be amplified is by adding cells directly to the nucleic acid amplification reaction mix and relying on the denaturation step of the amplification to lyse the cells and release the DNA.

The product of nucleic acid amplification reactions may be further characterized by one or more of the standard techniques that are well known in the art, including electrophoresis, restriction endonuclease cleavage patterns, oligonucleotide hybridization or ligation, and/or nucleic acid sequencing. When in hybridization techniques are used for cell identification purposes, a variety of probe labeling methods can be useful, including fluorescent labeling, radioactive labeling and non-radioactive labeling.

b. Protein-Based Analysis:

In addition to analysis of nucleic acids, microorganisms selected using the methods of the present invention can be characterized and identified based on the presence (or absence) of specific proteins directly. Such analysis can be based on the activity of the specified protein, e.g., through an enzyme assay or by the response of a co-cultured organisms, or by the mere presence of the specified protein (which can for instance be determined using immunologic methods, such as in situ immunofluorescent antibody staining).

Enzyme assays: By way of example, fluorescent or chromogenic substrate analogs can be included into the growth media (e.g., microtiter plate cultures), followed by incubation and screening for reaction products, thereby identifying cultures on a basis of their enzymatic activities.

Co-cultivation response: In some embodiments of the present invention, the activity of an enzyme carried by a microbial isolate can be assayed based on the response (or degree of response) of a co-cultured organism (such as a reporter organism).

A variety of methods can also be used for identifying microorganisms selected and isolated from a source environment by binding at least one antibody or antibody-derived molecule to a molecule, or more particularly an epitope of a molecule, of the microorganism.

Anti-microorganism protein antibodies may be produced using standard procedures described in a number of texts, including Harlow and Lane (Antibodies, A Laboratory Manual, CSHL, New York, 1988). The determination that a particular agent binds substantially only to a protein of the desired microorganism may readily be made by using or adapting routine procedures. One suitable in vitro assay makes use of the Western blotting procedure (described in many standard texts, including Harlow and Lane; Antibodies, A Laboratory Manual, CSHL, New York, 1988).

Shorter fragments of antibodies (antibody-derived molecules, for instance, FAbs, Fvs, and single-chain Fvs (SCFvs)) can also serve as specific binding agents. Methods of making these fragments are routine.

Detection of antibodies that bind to cells on an array of this invention can be carried out using standard techniques, for instance ELISA assays that provide a detectable signal, for instance a fluorescent or luminescent signal.

### The Polynucleotides and Polypeptides of the Invention

In another aspect of the present invention, the disclosure provides novel isolated nucleic acid molecules, nucleic acid molecules that interfere with these nucleic acid molecules, nucleic acid molecules that hybridize to these nucleic acid molecules, and isolated nucleic acid molecules that encode the same protein due to the degeneracy of the DNA code. Additional embodiments of the present application further include the polypeptides encoded by the isolated nucleic acid molecules of the present invention.

The polynucleotides and polypeptides of the present invention will preferably be “biologically active” with respect to either a structural attribute, such as the capacity of a nucleic acid to hybridize to another nucleic acid molecule, or the ability of a polypeptide to be bound by antibody (or to compete with another molecule for such binding). Alternatively, such an attribute may be catalytic and thus involve the capacity of the molecule to mediate a chemical reaction or response.

The polynucleotides and polypeptides of the present invention may also be recombinant. As used herein, the term recombinant means any molecule (e.g. DNA, peptide etc.), that is, or results, however indirect, from human manipulation of a polynucleotide or polypeptide.

Nucleic acid molecules or fragment thereof of the present invention are capable of specifically hybridizing to other nucleic acid molecules under certain circumstances. As used herein, two nucleic acid molecules are said to be capable of specifically hybridizing to one another if the two molecules are capable of forming an anti-parallel, double-stranded nucleic acid structure. A nucleic acid molecule is said to be the “complement” of another nucleic acid molecule if they exhibit complete complementarity. As used herein, molecules are said to exhibit “complete complementarity” when every nucleotide of one of the molecules is complementary to a nucleotide of the other. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Conventional stringency conditions are described by Sambrook et al., In: Molecular Cloning, A Laboratory Manual, 2nd Edition, Cold Spring Harbor Press, Cold Spring Harbor, N.Y. (1989), and by Haymes et al. In: Nucleic Acid Hybridization, A Practical Approach, IRL Press, Washington, D.C. (1985). Departures from complete complementarity are therefore permissible, as long as such departures do not completely preclude the capacity of the molecules to form a double-stranded structure. Thus, in order for a nucleic acid molecule or fragment of the present invention to serve as a primer or probe it need only be sufficiently complementary in sequence to be able to form a stable.

Appropriate stringency conditions which promote DNA hybridization are, for example, 6.0× sodium chloride/sodium citrate (SSC) at about 45° C., followed by a wash of 2.0×SSC at 50° C. The conditions are known to those skilled in the art, or can be found in Current Protocols in Molecular Biology, John Wiley & Sons, N.Y. 6.3.1-6.3.6 (1989). For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature at about 22° C., to high stringency conditions at about 65° C. Both temperature and salt may be varied, or either the temperature or the salt concentration may be held constant while the other variable is changed.

In a preferred embodiment, a nucleic acid of the present invention will specifically hybridize to one or more of the nucleic acid sequences set forth in the Sequence Listing or complements thereof under moderately stringent conditions, for example, at about 2.0×SSC and about 65° C.

In a particularly preferred embodiment, a nucleic acid of the present invention will include those nucleic acid molecules that specifically hybridize to one or more of the nucleic acid sequences set forth in the Sequence Listing or complements thereof under high stringency conditions.

In another embodiment, the present invention provides nucleotide sequences comprising regions that encode polypeptides. The encoded polypeptides may be the complete protein encoded by the gene represented by the polynucleotide, or may be fragments of the encoded protein. Preferably, polynucleotides provided herein encode polypeptides constituting a substantial portion of the complete protein, and more preferentially, constituting a sufficient portion of the complete protein to provide the relevant biological activity. Of particular interest are polynucleotides of the present invention that encode polypeptides involved in the production of biotoxins.

A subset of the nucleic acid molecules of this invention includes fragments of the disclosed polynucleotides consisting of oligonucleotides of at least 15, preferably at least 16 or 17, more preferably at least 18 or 19, and even more preferably at least 20 or more, consecutive nucleotides. Such oligonucleotides are fragments of the larger molecules having a sequence selected from the polynucleotide sequences in the Sequence Listing, and find use, for example, as interfering molecules, probes and primers for detection of the polynucleotides of the present invention.

In some embodiments, nucleic acid molecules that are fragments of these toxin-encoding nucleotide sequences are also encompassed by the present invention. A “toxin fragment” is intended to be a portion of the nucleotide sequence encoding a toxin protein. A fragment of a nucleotide sequence may encode a biologically active portion of a toxin protein, or it may be a fragment that can be used as a hybridization probe or PCR primer using methods disclosed below. Nucleic acid molecules that are fragments of a toxin nucleotide sequence comprise at least about 50, 100, 200, 300, 400, 500, 600, 700, 800, 900, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350 contiguous nucleotides, or up to the number of nucleotides present in a full-length toxin encoding nucleotide sequence disclosed herein depending upon the intended use. The term “contiguous nucleotides” is intended to mean nucleotide residues that are immediately adjacent to one another. Fragments of the nucleotide sequences of the present invention will encode protein fragments that retain the biological activity of the toxin protein and, hence, retain pesticidal activity. By “retains activity” is intended that the fragment will have at least about 30%, at least about 50%, at least about 70%, 80%, 90%, 95% or higher of the pesticidal activity of the toxin protein. Methods for measuring pesticidal activity are well known in the art. See, for example, Czapla and Lang (J. Econ. Entomol. 83:2480-2485, 1990); Andrews et al. (Biochem. J. 252:199-206, 1988); Marrone et al. (J. of Economic Entomology 78:290-293, 1985); and U.S. Pat. No. 5,743,477).

A fragment of a toxin-encoding nucleotide sequence that encodes a biologically active portion of a protein of the invention will encode at least about 15, 25, 30, 50, 75, 100, 125, 150, 175, 200, 250, 300, 350, 400, 450, 500, 550, 600, 650, 700, 750, 800, 850, 900, 950, 1000, 1050, 1100 contiguous amino acids, or up to the total number of amino acids present in a full-length toxin protein of the invention. In some embodiments, the fragment is a proteolytic cleavage fragment. For example, the proteolytic cleavage fragment may have an N-terminal or a C-terminal truncation of at least about 100 amino acids, about 120, about 130, about 140, about 150, or about 160 amino acids relative to any amino acid sequences set forth in the Sequence Listing. In some embodiments, the fragments encompassed herein result from the removal of the C-terminal crystallization domain, e.g., by proteolysis or by insertion of a stop codon in the coding sequence.

Also of interest in the present invention are variants of the polynucleotides provided herein. Such variants may be naturally occurring, including homologous polynucleotides from the same or a different species, or may be non-natural variants, for example polynucleotides synthesized using chemical synthesis methods, or generated using recombinant DNA techniques. With respect to nucleotide sequences, degeneracy of the genetic code provides the possibility to substitute at least one base of the protein encoding sequence of a gene with a different base without causing the amino acid sequence of the polypeptide produced from the gene to be changed. Hence, the DNA of the present invention may also have any base sequence that has been changed from any polynucleotide sequence in the Sequence Listing by substitution in accordance with degeneracy of the genetic code. References describing codon usage are readily publicly available.

The skilled artisan will further appreciate that changes can be introduced by mutation of the nucleotide sequences of the invention thereby leading to changes in the amino acid sequence of the encoded toxin proteins, without altering the biological activity of the proteins. Thus, variant isolated nucleic acid molecules can be created by introducing one or more nucleotide substitutions, additions, or deletions into the corresponding nucleotide sequence disclosed herein, such that one or more amino acid substitutions, additions or deletions are introduced into the encoded protein. Mutations can be introduced by standard techniques, such as site-directed mutagenesis and PCR-mediated mutagenesis. Such variant nucleotide sequences are also encompassed by the present invention.

For example, conservative amino acid substitutions may be made at one or more predicted, nonessential amino acid residues. A “nonessential” amino acid residue is a residue that can be altered from the wild-type sequence of a toxin protein without altering the biological activity, whereas an “essential” amino acid residue is required for biological activity. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g, tyrosine, phenylalanine, tryptophan, histidine).

As discussed elsewhere herein, delta-endotoxins generally have five conserved sequence domains, and three conserved structural domains (see, for example, de Maagd et al., 2001, supra). The first conserved structural domain consists of seven alpha helices and is involved in membrane insertion and pore formation. Domain II consists of three beta-sheets arranged in a Greek key configuration, and domain III consists of two antiparallel beta-sheets in “jelly-roll” formation (de Maagd et al., 2001, supra). Domains II and III are involved in receptor recognition and binding, and are therefore considered determinants of toxin specificity. Amino acid substitutions may be made in nonconserved regions that retain function. In general, such substitutions would not be made for conserved amino acid residues, or for amino acid residues residing within a conserved motif, where such residues are essential for protein activity. Examples of residues that are conserved and that may be essential for protein activity include, for example, residues that are identical between all proteins contained in an alignment of the amino acid sequences of the present invention and known toxin sequences. Examples of residues that are conserved but that may allow conservative amino acid substitutions and still retain activity include, for example, residues that have only conservative substitutions between all proteins contained in an alignment of the amino acid sequences of the present invention and known toxin sequences. However, one of skill in the art would understand that functional variants may have minor conserved or nonconserved alterations in the conserved residues.

Alternatively, variant nucleotide sequences can be made by introducing mutations randomly along all or part of the coding sequence, such as by saturation mutagenesis, and the resultant mutants can be screened for ability to confer toxin activity to identify mutants that retain activity. Following mutagenesis, the encoded protein can be expressed recombinantly, and the activity of the protein can be determined using standard assay techniques.

Using methods such as PCR, hybridization, and the like corresponding toxin sequences can be identified, such sequences having substantial identity to the sequences of the invention. See, for example, Sambrook and Russell (2001, supra.)

Polynucleotides of the present invention that are variants of the polynucleotides provided herein will generally demonstrate significant identity with the polynucleotides provided herein. Of particular interest are polynucleotide homologs having at least about 50% sequence identity, at least about 60% sequence identity, at least about 70% sequence identity, at least about 80% sequence identity, at least about 85% sequence identity, and more preferably at least about 90%, 95% or even greater, such as 96%, 97%, 98% or 99% sequence identity with any one of the polynucleotide sequences described herein.

The skilled artisan will further appreciate that once a novel toxin gene is identified, the nucleic acid molecules and fragments thereof corresponding to the novel toxin gene may then be used to identify the microbial strains or isolates in which the extrachromosomal genetic content naturally comprises a nucleic acid sequence identical to that of the novel toxin gene of interest. Such microbial strains or isolates can be readily identified by using the above-described nucleic acid molecules or fragments thereof to screen a microbial population. Screening of bacterial colonies by using PCR or DNA-based hybridization methods, antibody-based hybridization methods, among other well-known methods, is routine in the art.

Nucleic acid molecules and fragments thereof of the present invention may be employed to obtain other nucleic acid molecules from the same species. Such nucleic acid molecules include the nucleic acid molecules that have the complete coding sequence of a protein and promoters and flanking sequences of such molecules. In addition, such nucleic acid molecules include nucleic acid molecules that encode for other toxins or gene family members. Such molecules can be readily obtained by using the above-described nucleic acid molecules or fragments thereof to screen cDNA libraries or extrachromosomal DNA libraries obtained from toxin-producing microorganisms. Methods for generating such libraries are well known in the art.

Nucleic acid molecules and fragments thereof of the present invention may also be employed to obtain nucleic acid homologues. Such homologues include the nucleic acid molecules of different alleles within the same species or other organisms, including the nucleic acid molecules that encode, in whole or in part, toxin protein homologues of other organisms, sequences of genetic elements such as promoters and transcriptional regulatory elements. Such molecules can be readily obtained by using the above-described nucleic acid molecules or fragments thereof to screen cDNA libraries or extrachromosomal DNA libraries obtained from such microorganism species. Methods for generating such libraries are well known in the art. Such homologue molecules may differ in their nucleotide sequences from those found in one or more of the nucleotides in the Sequence Listing or complements thereof because complete complementarity is not needed for stable hybridization. The nucleic acid molecules of the present invention therefore also include molecules that, although capable of specifically hybridizing with the nucleic acid molecules may lack “complete complementarity.” In a particular embodiment, methods of 3′ or 5′ RACE may be used to obtain such sequences.

Any of a variety of methods known in the art may be used to obtain one or more of the above-described nucleic acid molecules. Automated nucleic acid synthesizers can be employed for this purpose. In lieu of such synthesis, the disclosed nucleic acid molecules can be used to define a pair of primers that can be used with the polymerase chain reaction to amplify and obtain any desired nucleic acid molecule or fragment, which is standard in the art.

Further, the degeneracy of the genetic code, which allows different nucleotide sequences to code for the same protein or peptide, is also known in the art.

In an aspect of the present invention, one or more of the nucleic acid molecules of the present invention differ in nucleotide sequence from those encoding a toxin polypeptide or fragment thereof selected from the group consisting of the nucleotide sequences in the Sequence Listing due to the degeneracy in the genetic code in that they encode the same protein but differ in nucleotide sequence.

Also provided in another further aspect of the present invention are one or more of the nucleic acid molecules that differ in nucleotide sequence from those encoding a toxin polypeptide or fragment thereof selected from the group consisting of the nucleotide sequences in the Sequence Listing due to fact that the different nucleotide sequences encode a polypeptide having one or more conservative amino acid residues. It is understood that genetic codons capable of coding for such conservative substitutions are well known in the art.

This invention also provides polypeptides that are encoded by the polynucleotides of the invention. It is known in the art that one or more amino acids in a sequence can be substituted with other amino acid(s), the charge and polarity of which are similar to that of the substituted amino acid, i.e. a conservative amino acid substitution, resulting in a biologically/functionally silent change. Conservative substitutes for an amino acid within the polypeptide sequence can be selected from other members of the class to which the amino acid belongs. Amino acids can be divided into the following four groups: (1) acidic (negatively charged) amino acids, such as aspartic acid and glutamic acid; (2) basic (positively charged) amino acids, such as arginine, histidine, and lysine; (3) neutral polar amino acids, such as serine, threonine, tyrosine, asparagine, and glutamine; and (4) neutral nonpolar (hydrophobic) amino acids such as glycine, alanine, leucine, isoleucine, valine, proline, phenylalanine, tryptophan, cysteine, and methionine.

Conservative amino acid changes within the native polypeptides' sequence can be made by substituting one amino acid within one of these groups with another amino acid within the same group. Biologically functional equivalents of the polypeptides or fragments thereof of the present invention can have about 10 or fewer conservative amino acid changes, more preferably about 7 or fewer conservative amino acid changes, and most preferably about 5 or fewer conservative amino acid changes. In a preferred embodiment of the present invention, the polypeptide has between about 5 and about 500 conservative changes, more preferably between about 10 and about 300 conservative changes, even more preferably between about 25 and about 150 conservative changes, and most preferably between about 5 and about 25 conservative changes or between 1 and about 5 conservative changes. The encoding nucleotide sequence will thus have corresponding base substitutions, permitting it to encode biologically functional equivalent forms of the proteins or fragments of the present invention.

In another aspect of the present invention, biotoxin polypeptides are also encompassed within the present invention. In an embodiment of this aspect, by “biotoxin polypeptide” is intended a polypeptide having an amino acid sequence comprising any one of the amino acid sequences set forth in the Sequence Listing. In some embodiments, the biotoxin polypeptides are encoded by a nucleic acid molecule including a nucleic acid sequence corresponding to any one of the nucleotide sequences in the Sequence Listing; or a nucleic acid sequence hybridizing under high stringency conditions to any one of the nucleotide sequences in the Sequence Listing, a complement thereof or a fragment of either; a nucleic acid sequence exhibiting 70% or greater sequence identity to any one of the nucleotide sequences in the Sequence Listing, a complement thereof or a fragment of either. In some embodiments, the biotoxin polypeptides exhibit 50% or greater sequence identity to any one of the amino acid sequences in the Sequence Listing.

As described in more detail elsewhere herein, biotoxin polypeptides can be effective in, for example, conferring pesticidal activity to a recombinant organism when expressed in such organism or in controlling a pest organism. Such biotoxin polypeptides typically contain at least one domain indicative of pesticidal activity. Examples of Pfam domains indicative of pesticidal activity that Applicants have identified in the biotoxin polypeptides described herein include Endotoxin_M (PF00555) domain (see, e.g., Li et al., Nature 353: 815-21, 1991; Cygler et al., J. Mol. Biol. 254 (3): 447-464, 1995; Ghosh et al., Acta Crystallogr. D 57: 1101-1109, 2001); Ricin_Blectin (PF00652) domain, Aerolysin (PF01117) domain (see, e.g., Howard et al., J. Bacteriol. 169: 2869-71, 1987; Parker et al., Nature 367: 292-5, 1994); Bac_thur_toxin (PF01338) domain (see, e.g., Li et al., J. Mol. Biol. 257:129-152, 1996); ETX_MTX2 (PF03318) domain (see, e.g., Thanabalu et al., Gene 170:85-89, 1996; Petit et al., J. Biol. Chem. 276:15736-15740, 2001); CBM_{—}6 (PF03422) domain (see, e.g., Henshaw et al., J. Biol. Chem. 279: 21552-21559, 2004); Binary_toxB (PF03495) domain (see, e.g., De Haan et al., Mol. Membr. Biol. 21: 77-92, 2004; Perelle et al., Infect. Immun. 61: 5147-56, 1993); ADPrib_exo_Tox (PF03496) domain (see, e.g., De Haan et al., 2004, supra; Perelle et al., 1993, supra); Endotoxin_C (PF03944) domain (see, e.g., Li et al., 1991, supra; Cygler et al., 1995, supra; Ghosh et al., 2001, supra); Endotoxin_N(PF03945) domain (see, e.g., Li et al., 1991, supra; Cygler et al., 1995, supra; Ghosh et al., 2001, supra), Toxin 10 (PF05431) domain (see, e.g., Humphreys et al., J. Invertebr. Pathol. 71:184-185, 1998); Botulinum HA-17 (PF05588) domain (see, e.g., Hutson et al., J. Biol. Chem. 271:10786-10792, 1996); CryBP1 (PF07029) domain (see, e.g., Dervyn et al., J. Bacteriol. 177:2283-2291, 1995; Zhang et al., J. Bacteriol. 179:4336-4341, 1997); PA14 (PF07691) domain (see, e.g., Rigden et al., Trends Biochem. Sci. 29:335-339, 2004); and Fve (PF09259) domain (see, e.g., Paaventhan et al., J Mol Biol. 332:461-470, 2003). More detailed description of specific Pfam domains can be found at various information sources, such as “www.sanger.ac.uk” or “pfam.janelia.org”. Further, specific polypeptides that are predicted to contain one or more indicative Pfam domains are described in great detail in the accompanying Sequence Listing. Thus, various practical applications of the biotoxin sequences in the sequence listing are immediately apparent to those of skill in the art based on their similarity to known sequences.

Fragments, biologically active portions, and variants thereof are also provided, and may be used to practice the methods of the present invention. “Fragments” or “biologically active portions” include polypeptide fragments comprising amino acid sequences sufficiently identical to any one of the amino acid sequences set forth in the Sequence Listing and that exhibit pesticidal activity. A biologically active portion of a toxin protein can be a polypeptide that is, for example, 10, 25, 50, 100 or more amino acids in length. Such biologically active portions can be prepared by recombinant techniques and evaluated for pesticidal activity. Methods for measuring pesticidal activity are well known in the art. See, for example, Czapla and Lang J. Econ. Entomot. 83:2480-2485 (1990); Andrews et al., Biochem. J. 252:199-206 (1988); Marrone et al., J. of Economic Entomology 78:290-293 (1985); WO2011009182A2; and U.S. Pat. No. 5,743,477. As used here, a fragment comprises at least 8 contiguous amino acids of any one of the amino acid sequences set forth in the Sequence Listing. The invention encompasses other fragments, however, such as any fragment in the protein greater than about 10, 20, 30, 50, 100, 150, 200, 250, 300, 350, 400, 400, 450, 500, 550, 600, 650, 700, 750, 800, 850, 900, 950, 1000, 1050, 1100, 1150, 1200, 1250, or 1300 amino acids.

As described elsewhere herein, by “variants” is intended proteins or polypeptides having an amino acid sequence that is at least about 60%, 65%, about 70%, 75%, about 80%, 85%, about 90%, 91%, 92%, 93%, 94%, 95%, 96%, 97%, 98% or 99% identical to any one of the amino acid sequences set forth in the Sequence Listing. Variants also include polypeptides encoded by a nucleic acid molecule that hybridizes to a nucleic acid molecule having a nucleotide sequence that comprises any one of the nucleotide sequences of the Sequence Listing, or a complement thereof, under stringent conditions. Variants include polypeptides that differ in amino acid sequence due to mutagenesis. Variant proteins encompassed by the present invention are biologically active, that is they continue to possess the desired biological activity of the native protein, which is retaining pesticidal activity. Methods for measuring pesticidal activity are well known in the art. See, for example, Czapla and Lang (1990, supra); Andrews et al., Biochem. J. (1988, supra); Marrone et al., (1985, supra); PCT Publication No. WO2011009182A2; and U.S. Pat. No. 5,743,477.

### Altered or Improved Variants

It is contemplated that DNA sequences of a toxin may be altered by various methods, and that these alterations may result in DNA sequences encoding proteins with amino acid sequences different than that encoded by a toxin of the present invention. This protein may be altered in various ways including amino acid substitutions, deletions, truncations, and insertions of one or more amino acids of the sequences set forth in the Sequence Listing, including up to about 2, about 3, about 4, about 5, about 6, about 7, about 8, about 9, about 10, about 15, about 20, about 25, about 30, about 35, about 40, about 45, about 50, about 55, about 60, about 65, about 70, about 75, about 80, about 85, about 90, about 100, about 105, about 110, about 115, about 120, about 125, about 130 or more amino acid substitutions, deletions or insertions.

Methods for such manipulations are generally known in the art. For example, amino acid sequence variants of a toxin protein can be prepared by mutations in the DNA. This may also be accomplished by one of several forms of mutagenesis and/or in directed evolution. In some aspects, the changes encoded in the amino acid sequence will not substantially affect the function of the protein. Such variants will possess the desired pesticidal activity. However, it is understood that the ability of a toxin to confer pesticidal activity may be improved by the use of such techniques upon the compositions of this invention. For example, one may express a toxin in host cells that exhibit high rates of base-misincorporation during DNA replication, such as XL-1 Red (Stratagene). After propagation in such strains, one can isolate the toxin DNA (for example by preparing plasmid DNA, or by amplifying by PCR and cloning the resulting PCR fragment into a vector), culture the toxin mutations in a non-mutagenic strain, and identify mutated toxin genes with pesticidal activity, for example by performing an assay to test for pesticidal activity.

Alternatively, alterations may be made to the protein sequence of many proteins at the amino or carboxy terminus without substantially affecting activity. This can include insertions, deletions, or alterations introduced by modern molecular methods, such as PCR, including PCR amplifications that alter or extend the protein coding sequence by virtue of inclusion of amino acid encoding sequences in the oligonucleotides utilized in the PCR amplification. Alternatively, the protein sequences added can include entire protein-coding sequences, such as those used commonly in the art to generate protein fusions. Such fusion proteins are often used to (1) increase expression of a protein of interest (2) introduce a binding domain, enzymatic activity, or epitope to facilitate either protein purification, protein detection, or other experimental uses known in the art (3) target secretion or translation of a protein to a subcellular organelle, such as the periplasmic space of Gram-negative bacteria, or the endoplasmic reticulum of eukaryotic cells, the latter of which often results in glycosylation of the protein.

Variant nucleotide and amino acid sequences of the present invention also encompass sequences derived from mutagenic and recombinogenic procedures such as DNA shuffling. With such a procedure, one or more different toxin protein coding regions can be used to create a new toxin protein possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between a toxin gene of the invention and other known toxin genes to obtain a new gene coding for a protein with an improved property of interest, such as an increased insecticidal activity. Strategies for such DNA shuffling are known in the art.

Domain swapping or shuffling is another mechanism for generating altered delta-endotoxin proteins. Domains II and III may be swapped between delta-endotoxin proteins, resulting in hybrid or chimeric toxins with improved pesticidal activity or target spectrum. Methods for generating recombinant proteins and testing them for pesticidal activity are well known in the art.

The skilled artisan will further appreciate that any of a variety of methods well known in the art may be used to obtain one or more of the above-described polypeptides. The polypeptides of the invention can be chemically synthesized or alternatively, polypeptides can be made using standard recombinant techniques in heterologous expression systems such as E. coli, yeast, insects, etc.

Bacterial genes quite often possess multiple methionine initiation codons in proximity to the start of the open reading frame. Often, translation initiation at one or more of these start codons will lead to generation of a functional protein. These start codons can include ATG codons. However, bacteria such as Bacillus sp. also recognize the codon GTG as a start codon, and proteins that initiate translation at GTG codons contain a methionine at the first amino acid. Furthermore, it is not often determined a priori which of these codons are used naturally in the bacterium. Thus, it is understood that use of one of the alternate methionine codons may also lead to generation of toxin proteins that encode pesticidal activity. These toxin proteins are encompassed in the present invention and may be used in the methods of the present invention.

### Information in the Sequence Listing

This specification contains nucleotide and polypeptide sequence information prepared using the program Patentln Version 3.5. The biotoxin sequences provided in the Sequence Listing are annotated to indicate one or several known homologs of the respective sequences. Some sequences contain “pfam” domains which are indicative of particular applications. The specific pfam domains are described in more detail by various sources, such as “www.sanger.ac.uk” or “pfam.janelia.org”. Thus, various practical applications of the biotoxin sequences in the sequence listing are immediately apparent to those of skill in the art based on their similarity to known sequences.

The biotoxin sequences provided in the Sequence Listing are annotated to indicate one or several known homologs of the respective sequences. Some sequences contain “Pfam” domains which are indicative of pesticidal activity. Pfam domains indicative of pesticidal activity that Applicants have identified in the biotoxin polypeptides described herein include Endotoxin_M (PF00555) domain, Ricin B lectin (PF00652) domain, Aerolysin (PF01117) domain, Bac_thur_toxin (PF01338) domain, ETX_MTX2 (PF03318) domain, CBM_{—}6 (PF03422) domain, Binary_toxB (PF03495) domain, ADPrib_exo_Tox (PF03496) domain, Endotoxin_C(PF03944) domain, Endotoxin_N(PF03945) domain, Toxin_{—10} (PF05431) domain, Botulinum HA-17 (PF05588) domain, CryBP1 (PF07029) domain, PA14 (PF07691) domain, and Fve (PF09259) domain. Some biotoxin sequences in the Sequence Listing are annotated in the “miscellaneous features” section with valuable applications of the respective sequences in, for example, conferring pesticidal activity to an organism, or in controlling a pest organism. Thus, various practical applications of the biotoxin sequences in the Sequence Listing are immediately apparent to those of skill in the art based on their similarity to known sequences.

Additional information of sequence applications comes from similarity to sequences in public databases. Entries in the “miscellaneous features” sections of the Sequence Listing labeled “NCBI GI:” and “NCBI Desc:” provide additional information regarding the respective sequences. In some cases, the corresponding public records, which may be retrieved from www.ncbi.nlm.nih.gov, cite publications with data indicative of uses of the annotated sequences.

From the disclosure of the Sequence Listing, it can be seen that the nucleotides and polypeptides of the inventions are sometimes useful, depending upon the respective individual sequence, to make transgenic organisms having one or more altered characteristics such as, for example, pesticidal activity. The present invention further encompasses nucleotides that encode the above described polypeptides, such as those included in the Sequence Listing, as well as the complements and/or fragments thereof, and include alternatives thereof based upon the degeneracy of the genetic code.

Some aspects of the present invention relate to an integrated strategy for isolation and identification of novel nucleotide sequences that encode biotoxins. By “novel nucleotide sequences” is intended nucleotide sequences that share less than about 30% sequence identity, preferably less than about 60% sequence identity, more preferably less than about 80% sequence identity, most preferably less than about 90%, 91%, 92%, 93%, 94%, 95%, 96%, 97%, 98%, or 99% sequence identity to any sequence in the database used for comparison.

Antibodies to the polypeptides of the present invention, or to variants or fragments thereof, are also encompassed. A variety of techniques and methods for producing antibodies are well known in the art (see, for example, Harlow and Lane (1988) Antibodies: A Laboratory Manual, Cold Spring Harbor Laboratory, Cold Spring Harbor, N.Y.; U.S. Pat. No. 4,196,265), and can be used to make an antibody according to the invention disclosed herein.

### Use of the Method of the Invention

The method described herein is useful for generating large metagenomic sequence datasets containing gene sequences of commercial value. Isolation and sequence of extrachromosomal nucleic acids specific to bacteria has several advantages over current methods for gene identification. First, since genes are identified by DNA sequence, this method is more likely to identify genes with lower DNA similarity to known genes than can readily be accomplished by hybridization. Second, since the extrachromosomal genomes of microbial strains will be a fraction of the total genome size (1-20%), it will be possible to rapidly sample the extrachromosomal genomes of many related or unrelated bacteria, and quickly identify interesting genes. Third, since much of strain-to-strain variation exists due to differences in extrachromosomal genetic content; this method will be very efficient at capturing the major diversity differences in bacterial groups. Furthermore, the efficiency of the method increases as the size of the existing sequence dataset increases. For any given microorganism, as the percent of novel clones detected can drop from 50% to 1%, the efficiency of the method disclosed herein may increase from 3-fold to 16-fold relative to sequencing the entire genome (for a 15 kb insert size).

Though only specific bacterial species are described herein, it is understood that the methods of the present invention can virtually be applied to all microorganisms that contain extrachromosomal DNA, including bacterial and fungal species. Extrachromosomal DNA can be isolated from these microorganisms and utilized in a method according to the present invention to identify novel toxin genes. Furthermore, it is understood that one may not need necessarily to isolate and/or purify the microbial cells in order to isolate and analyze its extrachromosomal DNA content; i.e. this method can be applied to samples from mixed populations, or of unknown origin, such as environmental samples.

Accordingly, some embodiments of the invention provide novel systems to screen mixed populations of microorganisms, enriched samples, or isolates thereof for polynucleotides encoding molecules having a toxin activity, so long as the microbial samples, strains, or isolates contain at least a toxin gene carried by extrachromosomal DNA. The method(s) of the invention allow the discovery of novel toxin molecules in vitro, and in particular novel toxin molecules derived from uncultivated or cultivated samples. Large populations of extrachromosomal DNA can be isolated, sequenced and screened using the method(s) of the invention. If so desired, the method(s) of the invention may allow one to screen and identify polynucleotides and the polypeptides encoded by these polynucleotides in vitro from a wide range of environmental samples.

In another embodiment, extrachromosomal nucleic acids of a plurality of isolates can be pooled after individual extractions to create a population of extrachromosomal nucleic acids that is suitable for subsequent sequencing, assembly, annotation, and gene identification. Alternatively, a plurality of microbial isolates can be combined prior to DNA extraction step, which also ultimately creates a population of extrachromosomal nucleic acids. Two or more of the populations of extrachromosomal nucleic acids can be pooled or combined to obtain a pooled population of extrachromosomal nucleic acids.

The microorganisms from which the extrachromosomal DNA may be isolated include prokaryotic microorganisms, such as Eubacteria and Archaebacteria, lower eukaryotic microorganisms such as fungi, algae and protozoa. The microorganisms may be cultured microorganisms or uncultured microorganisms obtained from environmental samples and include extremophiles, such as thermophiles, hyperthermophiles, psychrophiles and psychrotrophs. Of particular interest include but not limited to species of the bacterial genera Bacillus, Brevibacillus, Clostridia, Paenibacillus, Photorhabdus, Pseudomonas, Serratia, Streptomyces, or Xenorhabdus.

In one particular non-limiting exemplification, insecticidal proteins such as the Bacillus thuringiensis delta-endotoxin genes are often found on large extrachromosomal DNA molecules, and therefore can be rapidly discovered by using the screening methods disclosed herein. Thus isolation and sequencing of extrachromosomal DNA from Bacillus microorganisms, such as Bacillus thuringiensis is likely to lead to identification of novel delta-endotoxin genes. Such genes are likely to be valuable for the development of novel compositions and methods for controlling insect pests. In addition, many microorganisms of the genus Clostridia are also known to have large extrachromosomal plasmids, and some of these are known to contain virulence factors as well as toxins such as iota toxin (see, e.g., Perelle et al., Infect. Immun. 1993). Furthermore, it has been shown that the majority of gene variability of Clostridia microorganisms appears to occur due to plasmid content (see, e.g., Katayam et al., Mol. Gen. Genet. 1996). It is contemplated by the present inventors that the methods disclosed herein can be used to screen extrachromosomal DNA content of multiple Clostridia isolates to quickly capture a large amount of genetic diversity. In addition, there has been report of a homolog of delta-endotoxin gene present in Clostridia species (Barloy et al., J. Bacteriol. 1996). Thus applications of screening methods in accordance with the present invention can also be used in identifying novel bioxin genes in bacteria of this genus.

In addition, tumor-inducing and symbiotic plasmids are common in Agrobacterium and Rhizobium microorganisms (e.g., Van Larebeke et al., Nature 1974). Thus applications of a screening method in accordance with the present invention to the sequencing of bacterial tumor-inducing and symbiotic plasmids, especially those from known plant pathogens, is likely to identify novel genes involved in plant-pathogen interactions including genes involved in or required for both virulence and avirulence.

Further examples of microorganisms from which the extrachromosomal DNA content may be decoded using the methods provided herein include species of the bacterial genus Serratia where extrachromosomal DNA, such as pADAP plasmid of S. entomophila and pU143 plasmid of S. proteamaculans, are known to contain virulence associated regions (see, e.g., Hurst et al., Plasmid, 2011; Hurst et al., J. Bacteriol. 2000). Within a virulence-encoding region of the pADAP plasmid of S. entomophila, at least one gene cluster designated sepABC is important for S. entomophila pathogenicity. The Sep proteins are members of the Toxin complex (Tc) family of insecticidal proteins that were first identified in the nematode-associated bacteria Photorhabdus luminescens. The three Tc proteins Tc-A, Tc-B, and Tc-C typically combine to form a complex with insecticidal activity. A second pADAP virulence-encoding region contains 18 ORFs, the translated products of which have similarity to the Photorhabdus virulence cassettes (PVCs) that reside in the genome of the insecticidal bacterium P. luminescens TTO1. Therefore, the present inventors also contemplate that applications of screening methods in accordance with the present invention to decode the extrachromosomal genetic content of Serratia bacteria may identify novel sequences involved in or required for insecticidal activity as well as virulence and avirulence.

Furthermore, much of the diversity present in bacterial populations is present on extrachromosomal DNA content, including plasmids. Many of microbial plasmids are known to contain virulence factors, important for infectivity or severity of infection by bacteria pathogens. Correspondingly, it is likely that many of the proteins expressed by plasmid genomes are likely to have value as vaccines. For example, both plasmids pXO1 and pXO2 of Bacillus anthracis encode proteins required for pathogenesis during anthrax infection. For example, pXO2 encodes proteins that produce a protective capsule around the bacterium. The pXO1 plasmid encodes the three proteins of the anthrax toxin complex, lethal factor (LF), edema factor (EF), and the protective antigen (PA). The PA protein (protective antigen) forms the basis of a vaccine for anthrax. The present applicants contemplate that the rapid and efficient sequencing of bacterial plasmids by using a screening method disclosed herein will yield information with which one can create a database of proteins that might serve as effective vaccines.

### Use of the Molecules of the Invention

In one aspect of the invention, one may use one of many known methods to identify DNA sequences adjacent to polynucleotide sequences of interest. For example, one may further identify genomic regions that naturally surround a novel polynucleotide sequence in microbial cell. One may accomplish this by generating hybridization probes and screening an existing library of extrachromosomal DNA. Alternatively, one may generate a library of larger inserts (for example a cosmid library), and screen for clones likely to contain DNA adjacent to the novel polynucleotide sequence of interest. For example, one may clone and sequence regions flanking a known DNA by inverse PCR (Sambrook and Russell, supra). Another such method involves ligating linkers of known sequence to extrachromosomal DNA digested with restriction enzymes, then generating PCR product using an oligonucleotide homologous to the oligo linker, and an oligo homologous to the region of interest (e.g. the end sequence of a novel polynucleotide sequence of the invention). A kit for performing this procedure (GENOMEWALKER™, Clonetech) is available commercially.

For example, in a hybridization procedure, all or part of a toxin-encoding nucleotide sequences can be used to screen cDNA or genomic libraries. Methods for construction of such cDNA and genomic libraries are generally known in the art and are disclosed in Sambrook and Russell (2001, supra). The so-called hybridization probes may be genomic DNA fragments, cDNA fragments, RNA fragments, or other oligonucleotides, and may be labeled with a detectable group such as ³²P, or any other detectable marker, such as other radioisotopes, a fluorescent compound, an enzyme, or an enzyme co-factor. Probes for hybridization can be made by labeling synthetic oligonucleotides based on the known toxin-encoding nucleotide sequence disclosed herein. Degenerate primers designed on the basis of conserved nucleotides or amino acid residues in the nucleotide sequence or encoded amino acid sequence can additionally be used. The probe typically comprises a region of nucleotide sequence that hybridizes under stringent conditions to at least about 12, at least about 25, at least about 50, 75, 100, 125, 150, 175, 200, 250, 300, 350, or 400 consecutive nucleotides of toxin encoding nucleotide sequence of the invention or a fragment or variant thereof. Methods for the preparation of probes for hybridization are generally known in the art and are disclosed in Sambrook and Russell (2001, supra) herein incorporated by reference.

Hybridization of such sequences may be carried out using hybridization conditions under which a probe will hybridize to its target sequence to a detectably greater degree than to other sequences (e.g., typically at least 2-fold over background). Hybridization conditions are sequence-dependent and will be different in different circumstances. By controlling the stringency of the hybridization and/or washing conditions, target sequences that are 100% complementary to the probe can be identified (homologous probing). Alternatively, hybridization conditions can be adjusted to allow some mismatching in sequences so that lower degrees of similarity are detected (heterologous probing). Generally, a probe is less than about 1000 nucleotides in length, preferably less than 500 nucleotides in length, more preferably less than 200 nucleotides in length, and most preferably less than 100 nucleotides in length.

While many of the commercial uses of the resulting sequences can be apparent from direct inspection of the resulting sequences, one may perform additional steps to identify further commercial uses of the resulting sequences or genes.

### Conferring Pest Resistance to Crop Plants

In another aspect of the invention, methods are provided for the generation of transgenic organisms, particularly transgenic plants expressing a toxin that has pesticidal activity, which typically involves introducing a nucleic acid construct into an organism. For example, by “introducing” is intended to present to the plant the nucleic acid construct in such a manner that the construct gains access to the interior of a cell of the plant. The methods of the invention do not require that a particular method for introducing a nucleotide construct to a plant is used, only that the construct gains access to the interior of at least one cell of the plant. Methods described in details below by way of example may be utilized to generate transgenic plants, but the manner in which the transgenic plant cells are generated is not critical to this invention.

The transgenic plants of the invention may express one or more of the pesticidal sequences disclosed herein. In various embodiments, the transgenic plant further comprises one or more additional genes for insect resistance, for example, one or more additional genes for controlling coleopteran, lepidopteran, heteropteran, or nematode pests. It will be understood by one of skill in the art that the transgenic plant may comprise any gene imparting an agronomic trait of interest.

A variety of methods for introducing nucleic acid constructs into plants are known in the art including, but not limited to, stable transformation methods, transient transformation methods, and virus-mediated methods. Plants expressing a toxin may be subsequently isolated by common methods described in the art, for example by transformation of callus, selection of transformed callus, and regeneration of fertile plants from such transgenic callus. In such process, one may use any gene as a selectable marker so long as its expression in plant cells confers ability to identify or select for transformed cells.

### Expression Vectors

One or more of the polypeptides or fragments thereof encoded by the nucleic acid molecules of the present invention may be expressed in a transformed cell or transformed organism. For example, to use the sequences of the present invention or a combination of them or parts and/or mutants and/or fusions and/or variants of them, recombinant nucleic acid constructs may be prepared which comprise the polynucleotide sequences of the invention inserted into a vector, and which are suitable for transformation of plant cells. The construct can be made using standard recombinant DNA techniques and can be introduced to the species of interest by Agrobacterium-mediated transformation or by other means of transformation as referenced below. In addition, the microbial toxin sequences of the invention may be modified or codon-optimized to obtain or enhance expression of the corresponding polypeptide in host cells, e.g., plant cells. Typically a construct that expresses such a toxin polypeptide would contain a promoter to drive transcription of the gene, as well as a 3′ untranslated region to allow transcription termination and polyadenylation. The organization of such constructs is well known in the art. In some instances, it may be useful to engineer the gene such that the resulting peptide is secreted, or otherwise targeted within the plant cell. For example, the gene can be engineered to contain a signal peptide to facilitate transfer of the peptide to the endoplasmic reticulum. It may also be preferable to engineer the plant expression cassette to contain an intron, such that mRNA processing of the intron is required for expression.

The vector backbone may be any of those typically used in the field such as plasmids, viruses, artificial chromosomes, BACs, YACs, PACs and vectors such as, for instance, bacteria-yeast shuttle vectors, lambda phage vectors, T-DNA fusion vectors and plasmid vectors.

Typically, the construct comprises a vector containing a nucleic acid molecule of the present invention with any desired transcriptional and/or translational regulatory sequences such as, for example, promoters, UTRs, and 3′ end termination sequences. Vectors may also include, for example, origins of replication, scaffold attachment regions (SARs), markers, homologous sequences, and introns. The vector may also comprise a marker gene that confers a selectable phenotype on plant cells. The marker may preferably encode a biocide resistance trait, particularly antibiotic resistance, such as resistance to, for example, kanamycin, bleomycin, or hygromycin, or herbicide resistance, such as resistance to, for example, glyphosate, chlorsulfuron or phosphinothricin.

In some instances, recombinant DNA constructs can include heterologous transcriptional signals and/or translational initiation signals that are added to the protein-encoded DNA fragments so that such DNA fragments can subsequently be transcribed and translated. The addition of new transcriptional and translational signals can be achieved by a variety of techniques including those commonly known in the art. For example, PCR-based methods or standard recombinant DNA cloning techniques can be used to add transcriptional start signal, and add a new ATG initiation codon in-frame to the protein coding regions of the DNA fragments.

It will be understood that more than one regulatory region may be present in a recombinant vector, e.g., promoters, introns, enhancers, upstream activation regions, transcription terminators, and inducible elements. For example, a suitable enhancer is a cis-regulatory element (−212 to −154) from the upstream region of the octopine synthase (ocs) gene. Fromm et al., Plant Cell 1:977-984 (1989). Thus, more than one regulatory region can be operably linked to a nucleic acid sequence of interest.

Promoters which are known or are found to cause transcription of DNA in host cells, e.g, plant cells or microbial cells; can be used in the present invention. These promoters may be obtained from a variety of sources, such as microbes, plants and plant viruses. Preferably, the particular promoter selected should be capable of causing sufficient expression to result in the production of an effective amount of a protein to cause the desired phenotype. In addition to promoters known to cause transcription of DNA in plant cells, other promoters may be identified for use in the current invention by screening a plant cDNA library or microbial cDNA library for genes which are selectively or preferably expressed in the target tissues or cells.

The choice of promoters to be included depends upon several factors, including but not limited to efficiency, selectability, inducibility, desired expression level, and cell- or tissue-preferential expression. One of skill in the art can routinely modulate the expression of a sequence by appropriately selecting and positioning promoters and other regulatory regions relative to the sequence.

A vector or construct may also include a transit peptide. Incorporation of a suitable chloroplast transit peptide may also be employed. Translational enhancers may also be incorporated as part of the vector DNA. DNA constructs could contain one or more 5′ non-translated leader sequences which may serve to enhance expression of the gene products from the resulting mRNA transcripts. Such sequences may be derived from the promoter selected to express the gene or can be specifically modified to increase translation of the mRNA. Such regions may also be obtained from viral RNAs, from suitable eukaryotic or prokaryotic genes, or from a synthetic gene sequence.

Constructs or vectors may also include, with the coding region of interest, a nucleotide sequence that acts, in whole or in part, to terminate transcription of that region. For example, such sequences have been isolated including the Tr7 3′ sequence and the nos 3′ sequence, or the like.

If proper polypeptide production is desired, a polyadenylation region at the 3′-end of the coding region is typically included. The polyadenylation region may be derived from the natural gene, from a variety of other plant genes or microbial genes, or from T-DNA, and may be synthesized in the laboratory.

### Plant Transformation

Nucleic acid molecules of the present invention may be introduced into the genome or the cell of the appropriate host plant by a variety of techniques. Transformation techniques as well as protocols for introducing nucleotide sequences into plants may vary depending on the type of plant or plant cell, i.e., monocot or dicot, targeted for transformation. These techniques, able to transform a wide variety of higher plant species, are well known and described in the technical and scientific literature. Generation of transgenic plants or transgenic plant cells may be performed by one of several methods including, but not limited to, transformation of plant cells by injection, microinjection, electroporation of DNA, fusion of cells or protoplasts, PEG-mediated transformation, use of biolistics, and via T-DNA using Agrobacterium tumefaciens or Agrobacterium rhizogenes or other bacterial hosts, for example.

In addition, a number of non-stable transformation methods that are well known to those skilled in the art may be desirable for the present invention. Such methods include, but are not limited to, transient expression and viral transfection.

Methods for transformation of chloroplasts are known in the art. See, for example, Svab et al., Proc. Natl. Acad. Sci. USA (1990); Svab and Maliga, Proc. Natl. Acad. Sci. USA (1993); Svab and Maliga, EMBO J. (1993). The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride et al., Proc. Natl. Acad. Sci. USA (1994).

Seeds are obtained from the transformed plants and used for testing stability and inheritance. Generally, two or more generations are cultivated to ensure that the phenotypic feature is stably maintained and transmitted.

A person of ordinary skill in the art recognizes that after the expression cassette is stably incorporated in transgenic plants and confirmed to be operable, it can be introduced into other plants by sexual crossing. Any of a number of standard breeding techniques can be used, depending upon the species to be crossed.

It is also to be understood that two different transgenic plants can also be mated to produce offspring that contain two independently segregating added exogenous genes. Selfing of appropriate progeny can produce plants that are homozygous for both added exogenous genes. Back-crossing to a parental plant and out-crossing with a non-transgenic plant are also contemplated, as is vegetative propagation.

### Evaluation of Plant Transformation

Following introduction of heterologous foreign DNA into plant cells, the transformation or integration of heterologous gene in the plant genome can be confirmed by various methods such as analysis of nucleic acids, proteins and metabolites associated with the integrated gene.

PCR analysis is a rapid method, among others, to screen transformed cells, tissue or shoots for the presence of incorporated gene at the earlier stage before transplanting into the soil (Sambrook and Russell, Molecular Cloning: A Laboratory Manual. Cold Spring Harbor Laboratory Press, Cold Spring Harbor, N.Y. 2001). PCR can be carried out using oligonucleotide primers specific to the toxin gene of interest or Agrobacterium vector background, etc.

Plant transformation may be confirmed by Southern blot analysis of genomic DNA (Sambrook and Russell, 2001, supra). In general, total DNA is extracted from the transformant, digested with appropriate restriction enzymes, fractionated in an agarose gel and transferred to a nitrocellulose or nylon membrane. The membrane or “blot” is then probed with, for example, radiolabeled ³²P target DNA fragment to confirm the integration of introduced gene into the plant genome according to standard techniques (Sambrook and Russell, 2001, supra).

In Northern blot analysis, RNA is isolated from specific tissues of transformant, fractionated in a formaldehyde agarose gel, and blotted onto a nylon filter according to standard procedures that are routinely used in the art (e.g., Sambrook and Russell, 2001, supra). Expression of RNA encoded by the toxin is then tested by hybridizing the filter to a radioactive probe derived from a toxin, by methods known in the art (e.g., Sambrook and Russell, 2001, supra).

Western blot, biochemical assays and the like may be carried out on the transgenic plants to confirm the presence of protein encoded by the toxin gene by standard procedures (e.g., Sambrook and Russell, 2001, supra) using antibodies that bind to one or more epitopes present on the toxin protein.

As discussed above, a number of markers have been developed for use with plant cells, such as resistance to chloramphenicol, the aminoglycoside G418, hygromycin, or the like. Other genes that encode a product involved in chloroplast metabolism may also be used as selectable markers. Additionally, the genes disclosed herein are also useful as markers to assess transformation of bacterial cells or plant cells. Methods for detecting the presence of a transgene in a plant, plant organ (e.g., leaves, stems, roots, etc.), seed, plant cell, propagule, embryo or progeny of the same are well known in the art. In some embodiments, the presence of the transgene may be detected by testing for pesticidal activity.

Fertile plants expressing a toxin may be tested for pesticidal activity, and the plants showing optimal activity may be selected for further breeding. A variety of methods are available in the art to assay for pesticidal activity. Generally, the protein is mixed and used in feeding assays. See, for example Marrone et al., (1985, supra).

In principle, the methods and compositions according to the present invention can be deployed for any plant species. Monocotyledonous as well as dicotyledonous plant species are particularly suitable. The process is preferably used with plants that are important or interesting for agriculture, horticulture, for the production of biomass used in producing liquid fuel molecules and other chemicals, and/or forestry.

Thus, the invention has use over a broad range of plants, preferably higher plants pertaining to the classes of Angiospermae and Gymnospermae. Plants of the subclasses of the Dicotylodenae and the Monocotyledonae are particularly suitable. Dicotyledonous plants belong to the orders of the Aristochiales, Asterales, Batales, Campanulales, Capparales, Caryophyllales, Casuarinales, Celastrales, Cornales, Diapensales, Dilleniales, Dipsacales, Ebenales, Ericales, Eucomiales, Euphorbiales, Fabales, Fagales, Gentianales, Geraniales, Haloragales, Hamamelidales, Illiciales, Juglandales, Lamiales, Laurales, Lecythidales, Leitneriales, Magniolales, Malvales, Myricales, Myrtales, Nymphaeales, Papeverales, Piperales, Plantaginales, Plumbaginales, Podostemales, Polemoniales, Polygalales, Polygonales, Primulales, Proteales, Rafflesiales, Ranunculales, Rhamnales, Rosales, Rubiales, Salicales, Santales, Sapindales, Sarraceniaceae, Scrophulariales, Theales, Trochodendrales, Umbellales, Urticales, and Violales. Monocotyledonous plants belong to the orders of the Alismatales, Arales, Arecales, Bromeliales, Commelinales, Cyclanthales, Cyperales, Eriocaulales, Hydrocharitales, Juncales, Lilliales, Najadales, Orchidales, Pandanales, Poales, Restionales, Triuridales, Typhales, and Zingiberales. Plants belonging to the class of the Gymnospermae are Cycadales, Ginkgoales, Gnetales, and Pinales.

Suitable species may include members of the genus Abelmoschus, Abies, Acer, Agrostis, Allium, Alstroemeria, Ananas, Andrographis, Andropogon, Artemisia, Arundo, Atropa, Berberis, Beta, Bixa, Brassica, Calendula, Camellia, Camptotheca, Cannabis, Capsicum, Carthamus, Catharanthus, Cephalotaxus, Chrysanthemum, Cinchona, Citrullus, Coffea, Colchicum, Coleus, Cucumis, Cucurbita, Cynodon, Datura, Dianthus, Digitalis, Dioscorea, Elaeis, Ephedra, Erianthus, Erythroxylum, Eucalyptus, Festuca, Fragaria, Galanthus, Glycine, Gossypium, Helianthus, Hevea, Hordeum, Hyoscyamus, Jatropha, Lactuca, Linum, Lolium, Lupinus, Lycopersicon, Lycopodium, Manihot, Medicago, Mentha, Miscanthus, Musa, Nicotiana, Oryza, Panicum, Papaver, Parthenium, Pennisetum, Petunia, Phalaris, Phleum, Pinus, Poa, Poinsettia, Populus, Rauwolfia, Ricinus, Rosa, Saccharum, Salix, Sanguinaria, Scopolia, Secale, Solanum, Sorghum, Spartina, Spinacea, Tanacetum, Taxus, Theobroma, Triticosecale, Triticum, Uniola, Veratrum, Vinca, Vitis, and Zea.

The methods and compositions of the present invention are preferably used in plants that are important or interesting for agriculture, horticulture, biomass for the production of biofuel molecules and other chemicals, and/or forestry. Non-limiting examples include, for instance, Panicum virgatum (switchgrass), Sorghum bicolor (sorghum, sudangrass), Miscanthus giganteus (miscanthus), Saccharum sp. (energycane), Populus balsamifera (poplar), Zea mays (corn), Glycine max (soybean), Brassica napus (canola), Triticum aestivum (wheat), Gossypium hirsutum (cotton), Oryza sativa (rice), Helianthus annuus (sunflower), Medicago sativa (alfalfa), Beta vulgaris (sugarbeet), Pennisetum glaucum (pearl millet), Panicum spp., Sorghum spp., Miscanthus spp., Saccharum spp., Erianthus spp., Populus spp., Andropogon gerardii (big bluestem), Pennisetum purpureum (elephant grass), Phalaris arundinacea (reed canarygrass), Cynodon dactylon (bermudagrass), Festuca arundinacea (tall fescue), Spartina pectinata (prairie cord-grass), Arundo donax (giant reed), Secale cereale (rye), Salix spp. (willow), Eucalyptus spp. (eucalyptus), Triticosecale spp. (triticum—wheat X rye), Bamboo, Carthamus tinctorius (safflower), Jatropha curcas (Jatropha), Ricinus communis (castor), Elaeis guineensis (oil palm), Phoenix dactylifera (date palm), Archontophoenix cunninghamiana (king palm), Syagrus romanzoffiana (queen palm), Linum usitatissimum (flax), Brassica juncea, Manihot esculenta (cassaya), Lycopersicon esculentum (tomato), Lactuca saliva (lettuce), Musa paradisiaca (banana), Solanum tuberosum (potato), Brassica oleracea (broccoli, cauliflower, brusselsprouts), Camellia sinensis (tea), Fragaria ananassa (strawberry), Theobroma cacao (cocoa), Coffea arabica (coffee), Vitis vinifera (grape), Ananas comosus (pineapple), Capsicum annum (hot & sweet pepper), Allium cepa (onion), Cucumis melo (melon), Cucumis sativus (cucumber), Cucurbita maxima (squash), Cucurbita moschata (squash), Spinacea oleracea (spinach), Citrullus lanatus (watermelon), Abelmoschus esculentus (okra), Solanum melongena (eggplant), Papaver somniferum (opium poppy), Papaver orientale, Taxus baccata, Taxus brevifolia, Artemisia annua, Cannabis saliva, Camptotheca acuminate, Catharanthus roseus, Vinca rosea, Cinchona officinalis, Colchicum autumnale, Veratrum californica, Digitalis lanata, Digitalis purpurea, Dioscorea spp., Andrographis paniculata, Atropa belladonna, Datura stomonium, Berberis spp., Cephalotaxus spp., Ephedra sinica, Ephedra spp., Erythroxylum coca, Galanthus wornorii, Scopolia spp., Lycopodium serratum (Huperzia serrata), Lycopodium spp., Rauwolfia serpentine, Rauwolfia spp., Sanguinaria canadensis, Hyoscyamus spp., Calendula officinalis, Chrysanthemum parthenium, Coleus forskohlii, Tanacetum parthenium, Parthenium argentatum (guayule), Hevea spp. (rubber), Mentha spicata (mint), Mentha piperita (mint), Bixa orellana, Alstroemeria spp., Rosa spp. (rose), Dianthus caryophyllus (carnation), Petunia spp. (petunia), Poinsettia pulcherrima (poinsettia), Nicotiana tabacum (tobacco), Lupinus albus (lupin), Uniola paniculata (oats), bentgrass (Agrostis spp.), Populus tremuloides (aspen), Pinus spp. (pine), Abies spp. (fir), Acer spp. (maple), Hordeum vulgare (barley), Poa pratensis (bluegrass), Lolium spp. (ryegrass), Phleum pratense (timothy), and conifers. Of interest are plants grown for energy production, so called energy crops, such as cellulose-based energy crops like Panicum virgatum (switchgrass), Sorghum bicolor (sorghum, sudangrass), Miscanthus giganteus (miscanthus), Saccharum sp. (energycane), Populus balsamifera (poplar), Andropogon gerardii (big bluestem), Pennisetum purpureum (elephant grass), Phalaris arundinacea (reed canarygrass), Cynodon dactylon (bermudagrass), Festuca arundinacea (tall fescue), Spartina pectinata (prairie cord-grass), Medicago sativa (alfalfa), Arundo donax (giant reed), Secale cereale (rye), Salix spp. (willow), Eucalyptus spp. (eucalyptus), Triticosecale spp. (triticum—wheat X rye), and Bamboo; and starch-based energy crops like Zea mays (corn) and Manihot esculenta (cassava); and sucrose-based energy crops like Saccharum sp. (sugarcane) and Beta vulgaris (sugarbeet); and biofuel-producing energy crops like Glycine max (soybean), Brassica napus (canola), Helianthus annuus (sunflower), Carthamus tinctorius (safflower), Jatropha curcas (Jatropha), Ricinus communis (castor), Elaeis guineensis (African oil palm), Elaeis oleifera (American oil palm), Cocos nucifera (coconut), Camelina sativa (wild flax), Pongamia pinnata (Pongam), Olea europaea (olive), Linum usitatissimum (flax), Crambe abyssinica (Abyssinian-kale), and Brassica juncea.

### Use of the Molecules of the Invention in Making Recombinant Microbes:

General methods for employing microbial strains comprising a nucleic acid or polypeptide sequence according to the present invention, or a variant thereof, in pest control or in engineering other microorganisms as pesticidal agents are known in the art. See, for example U.S. Pat. Nos. 7,129,212; 7,056,888; 5,308,760; and 5,039,523.

For examples, the microbial strains, e.g. Bacillus species, containing a nucleic acid sequence of the present invention, or a variant thereof, or the microorganisms that have been genetically altered to contain a pesticidal gene sequence and protein may be used for protecting agricultural crops and products from pests. In one aspect of the invention, whole cells, i.e. unlysed cells, of a toxin (pesticide)-producing organism are treated with reagents that prolong the activity of the toxin produced in the cells when the cells are applied to the environment of target pest(s).

Alternatively, polypeptides having toxin-encoding sequences according to the present invention can be cloned and introduced in Pseudomonas spp., thus expressing the proteins and microencapsulating them in the bacterial cell wall. A variety of techniques suitable for production of bacterial toxins in Pseudomonas spp. are known in the art. Microencapsulated toxin could be used in spray applications alone or in rotations with B. thuringiensis-based insecticides containing other toxins.

Alternatively, a bio-pesticide can be produced by introducing a toxin-encoding sequence into a cellular host. Expression of the toxin gene results, directly or indirectly, in the intracellular production and maintenance of the bio-pesticide. In one aspect of this invention, these cells are then treated under conditions that prolong the activity of the toxin produced in the cell when the cell is applied to the environment of target pest(s). The resulting product retains the toxicity of the toxin. These naturally encapsulated pesticides may then be formulated in accordance with conventional techniques for application to the environment hosting a target pest, e.g., soil, water, and foliage of plants. See, for example U.S. Pat. No. 4,695,462; and the references cited therein. Alternatively, one may formulate the cells expressing a gene of this invention such as to allow application of the resulting material as a pesticide.

### Pesticidal Compositions

The polypeptides according to the present invention are normally applied in the form of compositions and can be applied to the crop area or plant to be treated, simultaneously or in succession, with other compounds and compositions. These compounds and compositions can be cryoprotectants, detergents, dormant oils, fertilizers, pesticidal soaps, polymers, surfactants, weed killers, and/or time-release or biodegradable carrier formulations that permit long-term dosing of a target area following a single application of the formulation. They can also be selective chemical bacteriocides, insecticides, herbicides, fungicides, microbicides, amoebicides, pesticides, nematocides, molluscicides, virucides, or mixtures of several of these preparations, if desired, together with further agriculturally acceptable carriers, surfactants or application-promoting adjuvants customarily employed in the art of formulation. Suitable carriers and adjuvants can be solid or liquid and correspond to the substances ordinarily employed in formulation technology, e.g. natural or regenerated mineral dispersants, substances, solvents, tackifiers, wetting agents, binders, or fertilizers. Likewise the formulations may be prepared into edible “baits” or fashioned into pest “traps” to permit feeding or ingestion by a target pest of the pesticidal formulation.

In some embodiments, methods of applying a pesticidal polypeptide or an agro-biochemical composition in accordance with the present invention, which contains at least one of the pesticidal polypeptides of the present invention, include leaf application, seed coating and soil application. The number of applications and the rate of application depend on the intensity of infestation by the corresponding pest.

The composition may be formulated as a powder, dust, pellet, granule, spray, emulsion, colloid, solution, or such like, and may be prepared by such conventional means as centrifugation, concentration, desiccation, extraction, filtration, homogenization, or sedimentation of a culture of cells comprising the polypeptide. In all such compositions that contain at least one such pesticidal polypeptide, the polypeptide may be present in a concentration of from about 1% to about 99% by weight.

Coleopteran, dipteran, lepidopteran, or nematode pests may be killed or reduced in numbers in a given area by the methods of the invention, or may be prophylactically applied to an environmental area to prevent infestation by a susceptible pest. Preferably the pest ingests, or is contacted with, a pesticidally-effective amount of the polypeptide. As disclosed above, a “pesticidally-effective amount” is intended as an amount of a pesticide or a pesticidal treatment which is necessary to obtain a reduction in the level of pest development and/or in the level of pest infection relative to that occurring in an untreated control. This amount will vary depending on such factors as, for example, the specific target pests to be controlled, the specific environment, location, plant, crop, or agricultural site to be treated, the environmental conditions, and the method, rate, concentration, stability, and quantity of application of the pesticidally-effective polypeptide composition. The formulations may also vary with respect to climatic conditions, environmental considerations, and/or frequency of application and/or severity of pest infestation.

The pesticidal compositions described herein may be made by formulating the microbial cell, spore suspension, bacterial crystal, or isolated protein component with the desired agriculturally-acceptable carrier. The compositions may be formulated prior to administration in an appropriate means such as lyophilized, freeze-dried, desiccated, or in an aqueous carrier, medium or suitable diluent, such as saline or other buffer. The formulated compositions may be in the form of a dust or granular material, or a suspension in oil (vegetable or mineral), or water or oil/water emulsions, or as a wettable powder, or in combination with any other carrier material suitable for agricultural application. Suitable agricultural carriers can be solid or liquid and are well known in the art. The term “agriculturally-acceptable carrier” covers all adjuvants, inert components, dispersants, surfactants, tackifiers, binders, etc. that are ordinarily used in pesticide formulation technology; these are well known to those skilled in pesticide formulation. The formulations may be mixed with one or more solid or liquid adjuvants and prepared by various means, e.g., by homogeneously mixing, blending and/or grinding the pesticidal composition with suitable adjuvants using conventional formulation techniques. Suitable formulations and application methods are described in, for example, U.S. Pat. Appl. No. US 20090087863A1.

The plants can also be treated with compositions of the invention that comprise one or more chemical compositions, including one or more herbicide, insecticides, or fungicides. Exemplary chemical compositions include herbicides (S-)Metolachlor, Alachlor, Amidosulfuron, Atrazine, Azimsulfuron, Beflubutamid, Bensulfuron, Bents zone, Benzobicyclon, Bispyribac, Bromacil, Bromoxynil, Butachlor, Butafenacil, Carfentrazone, Chloridazon, Chlorimuron-Ethyl, Chlorsulfuron, Clethodim, Clodinafop, Clopyralid, Cloransulam-Methyl, Cycloxydim, Cyhalofop, Daimuron, Desmedipham, Diclofop, Diflufenican, Diuron, Ethofumesate, Ethoxysulfuron, Fenoxaprop, Fentrazamide, Florasulam, Fluazifop, Fluazifop-butyl, Flucarbazone, Flufenacet, Flumioxazin, Fluometuron, Fluoroxypyr, Flupyrsulfuron, Fomesafen, Glufosinate, Glyphosate, Halosulfuron, Halosulfuron Gowan, Imazamox, Imazaquin, Imazethapyr, Imazosulfuron, Indanofan, Indaziflam, Iodosulfuron, Ioxynil, Isoproturon, Lenacil, Linuron, Mefenacet, Mesosulfuron, Mesotrione, Metamitron, Metazachlor, Metribuzin, Metsulfuron, MSMA, Nicosulfuron, Norflurazon, Oxadiargyl, Oxadiazone, Oxaziclomefone, Oxidemethon-methyl, Oxyfluorfen, Paraquat, Pendimethalin, Penoxsulam, Phenmedipham, Phenoxies, Picolinafen, Pinoxaden, Pirimicarb, Pretilachlor, Primisulfuron, Prometryn, Propanil, Propoxycarbazone, Propyzamide, Pyrasulfotole, Pyrazosulfuron, Pyributicarb, Pyriftalid, Pyrimisulfan, Pyrithiobac-sodium, Pyroxasulfon, Pyroxsulam, Quinclorac, Quinmerac, Quizalofop, Rimsulfuron, Saflufenacil, Sethoxydim, Simazine, Sulcotrione, Sulfosulfuron, Tefuryltrione, Tembotrione, Tepraloxydim, Thiacloprid, Thiamethoxam, Thidiazuron, Thiencarbazone, Thifensulfuron, Thiobencarb, Topramezone, Tralkoxydim, Triallate, Triasulfuron, Tribenuron, Trifloxysulfuron, Trifluralin, Trifluralin Ethametsulfuron, Triflusulfuron; Insecticides: (S-)Dimethenamid, (S-)Metolachlor, 4-[[(6-Chlorpyridin-3-yl)methyl](2,2-difluorethyl)amino]furan-2(5H)-on, Abamectin, Acephate, Acequinocyl, Acetamiprid, Acetochlor, Alachlor, Aldicarb, alpha-Cypermethrin, Avermectin, Bacillus thuriengiensis, Benfuracarb, beta-cyfluthrin, Bifenazate, Bifenthrin, Bromoxynil, Buprofezin, Cadusaphos, Carbaryl, Carbofuran, Cartap, Chlorpyrifos, Chlorpyriphos, Chromafenozide, Clopyralid, Clorphyriphos, Clothianidin, Cyanopyrafen, Cyaxypyr, Cyazypyr, Cyflumetofen, Cyfluthrin/beta-cyfluthrin, Cypermethrin, Deltamethrin, Diazinon, Dicamba, Dimethoate, Dinetofuran, Dinotefuran, Emamectin-benzoate, Endosulfan, Esfenvalerate, Ethiprole, Etofenprox, Fenamiphos, Fenbutatin-oxid, Fenitrothion, Fenobucarb, Fipronil, Flonicamid, Fluacrypyrim, Flubendiamide, Flufenacet, Foramsulfuron, Forthiazate, gamma and lambda Cyhalothrin, gamma Cyhalothrin, gamma/lambda Cyhalothrin, Gamma-cyhalothrin, Glufosinate, Glyphosate, Hexthiazox, Imidacloprid, Indoxacarb, Isoprocarb, Isoxaflutole, Lambda-cyhalothrin, Lambda-cyhalthrin, Lufenuron, Malathion, Mesotrione, Metaflumizone, Metamidophos, Methamidophos, Methiocarb, Methomyl, Methoxyfenozide, Monocrotophos, Novaluron, Organophosphates, Parathion, Profenophos, Pyrethroids, Pyridalyl, Pyriproxifen, Rynaxypyr, Spinodiclofen, Spinosad, Spinoteram, Spinotoram, Spirodiclofen, Spiromesifen, Spirotetramat, Sulfoxaflor, tau-Fluvaleriate, Tebupirimphos, Tefluthrin, Terbufos, Thiacloprid, Thiamethoxam, Thiocarb, Thiodicarb, Thriazophos, Tolfenpyrad, Triazophos, Triflumoron; Fungicides: Azoxystrobin, Boscalid, Carbendazim, Carpropamid, Chlorothalonil, Cyazofamid, Cyflufenamid, Cymoxanil, Cyproconazole, Cyprodinil, Diclocymet, Dimoxystrobin, EBDCs, Edifenphos, Epoxiconazole, Ethaboxam, Etridiazole, Fenamidone, Fenhexamid, Fenitropan, Fenoxanil, Fenpropimorph, Ferimzone, Fluazinam, Fludioxonil, Fluoxastrobin, Flutriafol, Fosetyl, Iprobenfos, Iprodione, Iprovalicarb, Isoprothiolane, Kresoxim-methyl, Metalaxyl, Metalaxyl/mefenoxam, Oxpoconazole fumarate, Pencycuron, Picoxystrobin, Probenazole, Prochloraz, Prothioconazole, Pyraclostrobin, Pyroquilon, Quinoxyfen, Quintozene, Simeconazole, Sulphur, Tebuconazole, Tetraconazole, Thiophanate-methyl, Thiram, Tiadinil, Tricyclazole, Trifloxystrobin, Vinclozolin, Zoxamide.

The pesticidal compositions of the present invention may be used in controlling one or more agronomically important pests including, but is not limited to, bacteria, fungi, insects, mites, nematodes, ticks, and the like. Insect pests include insects selected from the orders Anoplura, Coleoptera, Dermaptera, Diptera, Hemiptera, Homoptera, Hymenoptera, Isoptera, Lepidoptera, Mallophaga, Orthroptera, Siphonaptera, Thysanoptera, Trichoptera, etc., particularly Coleoptera, Diptera, and Lepidoptera.

Nematode pests of particular interest include parasitic nematodes such as root-knot, cyst, and lesion nematodes, including Heterodera spp., Globodera spp., and Meloidogyne spp.; particularly members of the cyst nematodes, including, but not limited to, Heterodera avenae (cereal cyst nematode); Heterodera glycines (soybean cyst nematode); Heterodera schachtii (beet cyst nematode); and Globodera pailida and Globodera rostochiensis (potato cyst nematodes). Lesion nematodes include Pratylenchus spp.

The pesticidal compositions of the present invention are preferably used in controlling insect pests of the major crops including, but not limited, Aceria tulipae, wheat curl mite; Acrosternum hilare, green stink bug; Agromyza parvicornis, corn blot leafminer; Agrotis ipsilon, black cutworm; Agrotis orthogonia, western cutworm; Anaphothrips obscrurus, grass thrips; Anthonomus grandis, boll weevil; Anticarsia gemmatalis, velvetbean caterpillar; Anuraphis maidiradicis, corn root aphid; Aphis gossypii, cotton aphid; Blissus leucopterus leucopterus, chinch bug; Bothyrus gibbosus, carrot beetle; Brevicoryne brassicae, cabbage aphid; Cephus cinctus, wheat stem sawfly; Chaetocnema pulicaria, corn flea beetle; Chilo partellus, sorghum borer; Colaspis brunnea, grape colaspis; Contarinia sorghicola, sorghum midge; corn leaf aphid; Cyclocephala borealis, northern masked chafer (white grub); Cyclocephala immaculata, southern masked chafer (white grub); Delia platura, seedcorn maggot; Delia ssp., Root maggots; Diabrotica longicornis barberi, northern corn rootworm; Diabrotica undecimpunctata howardi, southern corn rootworm; Diabrotica virgifera, western corn rootworm; Diatraea grandiosella, southwestern corn borer; Diatraea saccharalis, sugarcane borer; Diatraea saccharalis, surgarcane borer; Elasmopalpus lignosellus, lesser cornstalk borer; Eleodes, Conoderus, and Aeolus spp., wireworms; Empoasca fabae, potato leafhopper; Epilachna varivestis, Mexican bean beetle; Euschistus servus, brown stink bug; Feltia subterranea, granulate cutworm; Franklinkiella fusca, tobacco thrips; Helicoverpa zea, corn earworm; Helicoverpa zea, cotton bollworm; Heliothis virescens, cotton budworm; Homoeosoma electellum, sunflower moth; Hylemya coarctate, wheat bulb fly; Hylemya platura, seedcorn maggot; Hypera punctata, clover leaf weevil; Lissorhoptrus oryzophilus, rice water weevil; Lygus lineolaris, tarnished plant bug; Macrosiphum avenae, English grain aphid; Mamestra configurata, Bertha armyworm; Mayetiola destructor, Hessian fly; Melanoplus differentialis, differential grasshopper; Melanoplus femurrubrum, redlegged grasshopper; Melanoplus sanguinipes, migratory grasshopper; Melanotus spp., wireworms; Meromyza americana, wheat stem maggot; Myzus persicae, green peach aphid; Neolasioptera murtfeldtiana, sunflower seed midge; Nephotettix nigropictus, rice leafhopper; Ostrinia nubilalis, European corn borer; Oulema melanopus, cereal leaf beetle; Pectinophora gossypiella, pink bollworm; Petrobia latens, brown wheat mite; Phyllophaga crinita, white grub; Phyllotreta cruciferae, Flea beetle; Plathypena scabs, green cloverworm; Plutella xylostella, Diamond-back moth; Popillia japonica, Japanese beetle; Pseudaletia unipunctata, army worm; Pseudatomoscelis seriatus, cotton fleahopper; Pseudoplusia includens, soybean looper; Rhopalosiphum maidis; corn leaf aphid; Russian wheat aphid; Schizaphis graminum, greenbug; Sericothrips variabilis, soybean thrips; Sipha flava, yellow sugarcane aphid; Sitodiplosis mosellana, wheat midge; Sitophilus oryzae, rice weevil; Solenopsis milesta, thief ant; Sphenophorus maidis, maize billbug; Spodoptera exigua, beet armyworm; Spodoptera frugiperda, fall armyworm; Suleima helianthana, sunflower bud moth; Tetranychus cinnabarinus, carmine spider mite; Tetranychus turkestani, strawberry spider mite; Tetranychus urticae, twospotted spider mite; Thrips tabaci, onion thrips; Trialeurodes abutilonea, bandedwinged whitefly; Zygogramma exclamationis, sunflower beetle.

Throughout this disclosure, various information sources are referred to and incorporated by reference. The information sources include, for example, scientific journal articles, patent documents, textbooks, and World Wide Web browser-inactive page addresses. The reference to such information sources is solely for the purpose of providing an indication of the general state of the art at the time of filing. While the contents and teachings of each and every one of the information sources can be relied on and used by one of skill in the art to make and use embodiments of the invention, any discussion and comment in a specific information source should in no way be considered as an admission that such comment was widely accepted as the general opinion in the field.

The discussion of the general methods given herein is intended for illustrative purposes only. Other alternative methods and embodiments will be apparent to those of skill in the art upon review of this disclosure, and are to be included within the spirit and purview of this application.

It should also be understood that the following examples are offered to illustrate, but not limit, the invention.

### EXAMPLES

### Example 1

### Isolation of Microorganisms

First Isolation:

Microbial samples were collected from several sampling locations in the United States. Composite microbial samples for each sampling location were created from individual rhizosphere samples. Composites were created by taking 2 grams of rhizosphere soil from each individual sample and combining them in 50 mL Falcon tubes. Soils were homogenized after composites.

Composite microbial samples were subsequently used in a Bt enrichment procedure that involved growing the samples on a R&F® chromogenic plating medium containing a chromogenic substrate and inhibitory ingredients to inhibit growth of other bacteria, yeast and mold. This plating medium is routinely used to simultaneously identifying Bacillus cereus and B. thuringiensis cells from a mixed sample (Catalogue No. M-0400, R&F Products). This highly selective medium typically can help identify only B. cereus and B. thuringiensis isolates as blue colonies, while other Bacillus species either form white colonies or do not grow. Blue colonies, i.e. B. cereus and B. thuringiensis, were individually picked into 96-well cell culture plates containing 150 μL/well of 2YT medium and incubated at 30° C. overnight. These isolation plates were pin-tooled to create 2 new 96-well plates (replicates) and archived with 20% glycerol at −80° C.

Second Isolation:

1 gram of composite soil was placed into 10 mL LB medium supplemented with 0.25M sodium acetate and incubated at 30° C. on a shaker for 4 hours. Subsequently, these incubations were serially diluted or directly plated onto R&F® chromogenic plating medium, followed by incubation at 30° C. for 24 hours. Blue colonies, i.e. B. cereus and B. thuringiensis, were selected, incubated and archived as described above.

An initial 96-well plate containing Bt enrichment isolates was submitted to confirm the identity of the isolates via 16S rRNA sequencing. As described in details below, this initial 16S sequencing was done to validate enrichment and isolation methods and verify that the isolates recovered were Bacillus sp.

Bacterial Cell Lysis and Acquiring 16S rRNA Sequence Information

A 20 μL aliquot of cell suspension was transferred to a 96-well PCR plate containing 20 μl of a 2× lysis buffer (100 mM Tris HCL, pH 8.0, 2 mM EDTA, pH 8.0, 1% SDS, 400 μg/ml Proteinase K). Lysis conditions were as follows: 55° C. for 30 minutes, 94° C. for 4 minutes. An aliquot of the lysis product was used as the source of template DNA for PCR amplification. The 16S rRNA sequence was amplified via PCR using M13-27F (SEQ ID NO: 207) and 1492R M13-tailed (SEQ ID NO: 208) primers.

For amplification of 16S rRNA region, each PCR mixture was prepared in a 20 final volume reaction containing 4 μl from the bacterial lysis reaction, 2 uM of each primer (27F or 1492R), 6% Tween-20, and 10 μl of 2× ImmoMix (Bioline USA Inc, Taunton, Mass.). PCR conditions were as follows: 94° C. for 10 minutes; 94° C. for 30 seconds, 52° C. for 30 seconds, 72° C. for 75 seconds for 30 cycles; 72° C. for 10 minutes. A 2-p. 1 aliquot of the PCR product was run on a 1.0% agarose gel to confirm a single band of the expected size. Positive bands were purified and submitted for PCR sequencing. Sequencing was performed in the forward and reverse priming directions by the J. Craig Venter Institute in San Diego, Calif. using 454 technologies.

Homology searching for the determined nucleotide sequence was conducted using the DDBJ/GenBank/EMBL database. Sequence identity and similarity were also determined using GenomeQuest™ software (Gene-IT, Worcester Mass. USA). The sequence analysis results revealed that of the 92 Bt enrichment isolates, 91 isolates have an 16S rRNA gene sharing at least 98% sequence identity to that of B. cereus and/or B. thuringiensis strains previously identified. These results confirmed that the intended Bacillus cell populations were recovered from the selection step on R&F® chromogenic plating medium. Based on the observation that a large majority of blue colonies grown on R&F® chromogenic plating medium were indeed B. cereus and/or B. thuringiensis, 16S sequencing step can be made an optional step in subsequent selections of B. cereus and/or B. thuringiensis isolates.

Whenever phylogenetic reconstruction was needed, nucleotide sequences were aligned in Bioedit (located on the World Wide Web at www.mbio.ncsu.edu/bioedit/bioedit.html) followed by manual refinement. Phylogenetic trees were constructed in PHYML (located on the World Wide Web at pbil.univ-lyonl.fr/software/phyml_multi/) using maximum likelihood, HKY substitution model and the default settings. Branch support was obtained by bootstrapping (100 replicates).

### Example 2

### Purification of Extrachromosomal DNA from Mixed Populations of Microbial Isolates

An improved procedure for bacterial cell lysis was developed and optimized as follows.

A subset of Bt enrichment isolates were selected to verify the efficacy of cell lysis and the extrachromosomal DNA extraction method. Preparation of extrachromosomal DNA from the Bt enriched isolates was performed by essentially following a procedure described in Andrup et al. (Plasmid 59:139-143, 2008), with some modifications. For each isolate, a 7 mL 2×YT culture was inoculated with 50 μl, pre-culture, followed by an overnight incubation (12-16 hours) at 30° C. on a rotary shaker (200 rpm). Cells were pelleted at 3250×g for 30 minutes at 4° C., and resuspended in 100 μL, of extraction buffer (15% [wt/vol] sucrose, 40 mM Tris, 2 mM EDTA, pH7.9) by gently pipetteting the cell suspension up and down a few times. Cells were lysed by addition of 200 μL of lysing solution (3% [wt/vol] SDS, 50 mM Tris, pH 12.5). The lysate was heated at 60° C. for 30 minutes followed by the addition of 20 μL of Proteinase K (20 mg/mL, Finnzymes, Thermo Scientifics). The solution was mixed by inversion several times and incubated at 37° C. for 60 minutes. One milliliter of phenol-chloroform-isoamyl alcohol (25:24:1) was added, and the solution was inverted several times. After centrifugation 8000×g for 7 minutes, each extraction typically yielded ˜250 μl, of upper aqueous layer, which was transferred to a new tube. A 10 μL aliquot of the aqueous solution was subjected to electrophoresis to approximate the quantity of extrachromosomal DNA and contaminant genomic DNA, if any. Contaminant RNAs, which could generally interfere with subsequent pulsed-field gel electrophoresis (PFGE) step, was removed by the addition of 1 μL (10 mg/mL) of RNase (Fermentas). Pulsed-field gel electrophoresis (PFGE) was used to separate high-molecular weight nucleic acids. Approximately 40 μL of the aqueous solution from the DNA extraction step was mixed with 20 μL of melted agarose before being loaded into each well of a 1% agarose gel. The gel was run for 16 hours in 0.96×TAE buffer. Gel conditions were as follows: initial switch time was 5 seconds; final switch time was 20 seconds; 6 volts/cm, 120° angle, 300-350 mA during run. Standards were Epigene Bac tracker, Lambda midrange, and Lamba ladder (New England Biolabs). The gel was post-stained with ethidium bromide (1 μg/mL) and visualized under UV illumination. Visualization confirmed that isolates possessed extrachromosomal DNA, many with sizes greater than 100 kb.

Preparation of Extrachromosomal DNA Using QIAGEN® Reagents.

QIAGEN's large construct kit was used to extract extrachromosomal DNA from Bt isolates in attempts to remove genomic DNA from extrachromosomal DNA. Two approaches were attempted; (1) the QIAGEN® protocol was followed as recommended by manufacturer, (2) a modified cell lysis procedure was deployed to aid in lysing the Gram-positive Bacillus cells (because the original QIAGEN® protocol was developed for E. coli, a Gram negative bacterium).

Protocol 1: QIAGEN® protocol was followed as recommended. Incubation step (step 5 of QIAGEN® protocol) was 5 minutes at room temperature, followed by 1.5 hours on ice prior to neutralization step.

Protocol 2: Step 5 of QIAGEN® protocol was modified to be more rigorous for lysing Bacillus cells. This included a 30-minute incubation at 60° C. in a water bath and a 60 minute incubation at 37° C., with the addition of 250 μg/mL proteinase K.

Two hundred Bt enrichment isolates were grown individually in 5 mL Miller's LB each for 16 hours at 30° C. on a rotary shaker (200 rpm). Following incubation, individual cultures were combined to create a 1 L composite culture. 500 mL of this composite culture was pelleted and resuspended following the QIAGEN® large construct protocol. The modified lysis procedure for Bacillus cells was used in place of the recommended QIAGEN® step 5. The remaining steps were followed as recommended by the manufacturer.

Following extraction, final pellet from 2^{nd} ethanol precipitation step (QIAGEN® step 19) was resuspended in 500 μL of TE buffer (pH 8.5) and quantified fluorometrically via a Qubit® fluorometer (Invitrogen). 10 μL of each extraction were run on a 1.0% agarose gel. Visual assessment of the gel results revealed that the extrachromosomal DNA extracted using the improved procedure (i.e. Protocol 2) was presented on the gel as sheared DNA ranging in size from 0.5 to 30-Kb. By contrast, a control extraction that was performed following the exact recommendation of manufacturer (i.e. Protocol 1) yielded no DNA.

### Example 3

### Metagenomic Sequence Dataset Buildup: High-Throughput Sequencing, Sequence Assembly and Annotation

A pool of extrachromosomal nucleic acids purified from 200 Bacillus sp. isolates was shot-gun sequenced, assembled and annotated by using procedures described in PCT Patent Publication No. WO2010115156A2. The DNA template was subjected to a single lane of an Illumina Genome Analyzer IIx (GAIIx) platform according to the manufacturer's recommended conditions. Approximately 2 Gbp of 75 by paired-end reads were generated. The average insert size was ˜200 bp. Sequence assembly was then carried out using CLC Genomics Workbench de-novo assembler (CLC Bio), using default parameters. A total of 28,098 contigs with a total length of 18.3 Mbp and an N50 value of 702 by was assembled.

In a parallel sequencing experiment, the DNA template was also subjected to a single lane of an Illumina HiSeq 2000 Sequencing system, generating approximately 15 Gbp of 75 by paired-end reads with an average insert size of 200 bp. Sequence assembly was then carried out using CLC Genomics Workbench de-novo assembler (CLC Bio), using default parameters. A total of 47,551 contigs with a total length of 35.9 Mbp and an N50 value of 873 by was assembled.

The quality of the sequence data was significantly improved between the 2 data sets, with the HiSeq data providing greater coverage and generating more full-length sequences.

The remaining contigs of approximately 35 Mbp, i.e. the assembled contigs that did not show significant sequence similarity with the Bt toxin database and presumably represent other parts of the extrachromosomal DNA content, was also run through the prokaryotic annotation pipeline as described below.

Coding gene sequences were predicted from assembled contigs using an approach that combined evidence from multiple sources using the Evigan consensus gene prediction method as described previously by Liu et al. [Bioinformatics, March 1; 24(5):597-605, 2008]. All candidate ORFs on a metagenomic sequence read were first predicted based on stop codons found on all six frames and allowing for run-on in order to include partial ORFs. Candidate ORF translations were then annotated using Blastp searches against the NCBI non-redundant protein database and FastHMM (at microbesonline.org/fasthmm/) searches against Pfam (Finn et al., Nucleic Acids Res. 2008) and Superfamily (see, e.g., Inskeep et al., PLoS, 2010) domain databases. De novo ORF predictions were also made using 3 prokaryotic gene finding tools: Glimmer [Delcher et al., Bioinformatics, March 15; 23(6):673-9, 2007], Prodigal (at compbio.oml.gov/prodigal/), and Metagene [Brunet et al., Proc Natl Acad Sci USA, March 23; 101(12):4164-9, 2004]. The evidence from the blast/FastHMM searches and de novo gene finders was then combined in an unsupervised manner using Evigan. Since the start sites predicted by Evigan do not necessarily correspond to start codons, the predicted ORFs were extended upstream to the closest start codon in the same coding frame. The consensus gene prediction was performed by first binning contigs based on GC content and then running Evigan on each 10,000 contig bin separately.

### Example 4

### Use of Metagenomic Sequence Dataset to Rapidly Identify Novel Toxin Genes

Contigs resulting from the assembly and annotation process as described in Example 3 were then tested for presence of polynucleotide sequences encoding novel endotoxins by comparing the sequences against a database consisting of known endotoxins using the BLASTX algorithm. The analysis of the assembled and annotated sequences identified several genes belonging to many major classes of Bt toxins including Cry, VIP and Cyt genes. In total, 47 full-length and 56 partial novel toxin genes were identified along with many toxin genes previously discovered.

TABLE 1
Biotoxin-encoding sequences identified by the method of the invention.
Sequence identity was determined for each of the amino acid sequences using GenomeQuest ™
software with default settings. Exemplary functional homologs of each of the polypeptides are
provided. Other known homologs of the respective sequences are also provided in the
accompanying Sequence Listing.

| | | | | | Polynucleotide | Polypeptide |
|---|---|---|---|---|---|---|
| Gene ID | Length | Exemplary homologs | % identity | Toxin class | SEQ ID | SEQ ID |
| SG1METG47190 | Partial | GI: 229100569, C2VLX5 | 62.00 | Cry | 1 | 2 |
| SG1METG47191 | Partial | GI: 228911986, C3IBA6 | 66.00 | Cry | 3 | 4 |
| SG1METG47195 | Full-length | US20110263488 (SEQ ID | 81.03 | Cry | 5 | 6 |
| | | NO: 0004), US20080070829 | | | | |
| | | (SEQ ID NO: 0004) | | | | |
| SG1METG47207 | Partial | WO2011014749 (SEQ ID | 84.55 | Cry | 7 | 8 |
| | | NO: 0043) | | | | |
| SG1METG47218 | Partial | WO2010099365 (SEQ ID | 57.04 | Cry | 9 | 10 |
| | | NO: 0023, WO2010099365 | | | | |
| | | (SEQ ID NO: 0109), | | | | |
| | | WO2010099365 (SEQ ID | | | | |
| | | NO: 0110) | | | | |
| SG1METG47229 | Partial | U.S. Pat. No. 7,378,499 (SEQ ID | 45.36 | Cry | 11 | 12 |
| | | NO: 0044), U.S. Pat. No. 7,378,499 (SEQ | | | | |
| | | ID NO: 0050), WO2004003148 | | | | |
| | | (SEQ ID NO: 0050) | | | | |
| SG1METG47230 | Partial | YP_001642495, A9VV88, | 87.93 | Cry | 13 | 14 |
| | | ABY46520 | | | | |
| SG1METG47239 | Partial | GI: 228911387, C3I9T3 | 100.00 | Cry | 15 | 16 |
| SG1METG47244 | Partial | U.S. Pat. No. 7,452,700 (SEQ ID | 61.82 | Cry | 17 | 18 |
| | | NO: 0002), U.S. Pat. No. 7,329,736 (SEQ | | | | |
| | | ID NO: 0002) | | | | |
| SG1METG47245 | Partial | GI: 229100569, C2VLX5 | 62.00 | Cry | 19 | 20 |
| SG1METG47248 | Partial | WO2010099365 (SEQ ID | 77.53 | Cry | 21 | 22 |
| | | NO: 0072) | | | | |
| SG1METG47249 | Partial | ABY4652 | 91.98 | Cry | 23 | 24 |
| SG1METG47256 | Partial | GI: 48727548, A9UJY9 | 36.00 | Cry | 25 | 26 |
| SG1METG47260 | Partial | WO2010102172 (SEQ ID | 69.79 | Cry | 27 | 28 |
| | | NO: 0014) | | | | |
| SG1METG47261 | Partial | AZV31886, CN102417538 | 85.19 | Cry | 29 | 30 |
| | | (SEQ ID NO: 0001), | | | | |
| | | CN102417538 (SEQ ID | | | | |
| | | NO: 0002) | | | | |
| SG1METG47263 | Full-length | GI: 51090236, Q6BE06 | 50.00 | Cry | 31 | 32 |
| SG1METG47265 | Partial | JP2011526150 (SEQ ID | 53.57 | Cry | 33 | 34 |
| | | NO: 0197, JP2011526150 (SEQ | | | | |
| | | ID NO: 0198), WO2009158470 | | | | |
| | | (SEQ ID NO: 0268) | | | | |
| SG1METG47269 | Full-length | ZP_04069196, Q8KNU9. | 100.00 | Cyt | 35 | 36 |
| | | AOG39339 | | | | |
| SG1METG47272 | Partial | C3IW20, ZP_04069274, | 100.00 | Cry | 37 | 38 |
| SG1METG47321 | Partial | AXU72358, WO2009158470 | 70.01 | Cry | 39 | 40 |
| | | (SEQ ID NO: 0069) | | | | |
| SG1METG47324 | Full-length | US20100298207 (SEQ ID | 88.14 | Vip | 41 | 42 |
| | | NO: 0071) | | | | |
| SG1METG47325 | Full-length | WO2010099365 (SEQ ID | 75.91 | Vip | 43 | 44 |
| | | NO: 0070) | | | | |
| SG1METG47331 | Partial | GI: 51090239, Q6BE04 | 60.00 | Cry | 45 | 46 |
| SG1METG47332 | Full-length | GI: 17385650, Q8VUK9 | 39.00 | Cry | 47 | 48 |
| SG1METG47362 | Partial | GI: 228911944, C3IB67 | 100.00 | Cry | 49 | 50 |
| SG1METG47247 | Full-length | GI: 8928022, Q45729 | 36.00 | Cry | 51 | 52 |
| SG1METG47215 | Full-length | GI: 228937010, C3GC23 | 41.00 | Clostridium/ | 53 | 54 |
| | | | | epsilon toxin | | |
| SG1METG192243 | Partial | GI: 228918263, C3HSG6 | 64.00 | Clostridium/ | 55 | 56 |
| | | | | epsilon toxin | | |
| SG1METG186283 | Full-length | US20060191034 (SEQ ID | 81.04 | Clostridium/ | 57 | 58 |
| | | NO: 0006) | | epsilon toxin | | |
| SG1METG185109 | Partial | gi228918255, C3HSF9 | 34.00 | Clostridium/ | 59 | 60 |
| | | | | epsilon toxin | | |
| SG1METG203806 | Full-length | gi228918255, C3HSF9 | 34.00 | Clostridium/ | 61 | 62 |
| | | | | epsilon toxin | | |
| SG1METG215010 | Full-length | gi228918255, C3HSF9 | 31.00 | Clostridium/ | 63 | 64 |
| | | | | epsilon toxin | | |
| SG1METG217783 | Full-length | GI: 228949431, C3FB42 | 59.00 | Clostridium/ | 65 | 66 |
| | | | | epsilon toxin | | |
| SG1METG47259 | Partial | AEM22374 | 98.92 | Cry | 67 | 68 |
| SG1METG47235 | Partial | US20100017914 (SEQ ID | 99.88 | Cry | 69 | 70 |
| | | NO: 0078) | | | | |
| SG1METG47198 | Partial | AAP94035, WO2007147096 | 99.66 | Cry | 71 | 72 |
| | | (SEQ ID NO: 0004) | | | | |
| SG1METG47359 | Partial | Q2HWE8, BAE79727 | 100.00 | Cry | 73 | 74 |
| SG1METG47296 | Full-length | C3IVB1 | 100.00 | Cry | 75 | 76 |
| SG1METG47286 | Partial | AAX20050, CAD30095 | 100.00 | Cry | 77 | 78 |
| SG1METG47287 | Full-length | ZP_04069644, Q7AL73, | 100.00 | Cyt | 79 | 80 |
| | | AXW72396 | | | | |
| SG1METG47231 | Full-length | ZP_04069272, Q29Y56, | 100.00 | Cyt | 81 | 82 |
| | | Q7AL78 | | | | |
| SG1METG47319 | Full-length | ZP_04069020, Q3F161, | 100.00 | Cry | 83 | 84 |
| | | ZP_00738423 | | | | |
| SG1METG47320 | Full-length | ZP_04069019, Q3F160, | 100.00 | Cry | 85 | 86 |
| | | ZP_00738424 | | | | |
| AGRMET1T167125 | Partial | US20110263488 (SEQ ID | 74.65 | Cry | 87 | 88 |
| | | NO: 0004), US20080070829 | | | | |
| | | (SEQ ID NO: 0004), | | | | |
| | | AOG39300 | | | | |
| AGRMET1T140198 | Partial | AZE84673, WO2011014749 | 84.76 | Cry | 89 | 90 |
| | | (SEQ ID NO: 0043) | | | | |
| AGRMET1T166163 | Partial | AZE84673, WO2011014749 | 79.17 | Cry | 91 | 92 |
| | | (SEQ ID NO: 0043) | | | | |
| AGRMET1T218423 | Full-length | WO2007027776 (SEQ ID | 34.67 | Cry | 93 | 94 |
| | | NO: 0001), WO2007027776 | | | | |
| | | (SEQ ID NO: 0007), | | | | |
| | | WO2007027776 (SEQ ID | | | | |
| | | NO: 0009) | | | | |
| AGRMET1T218946 | Full-length | U.S. Pat. No. 7,572,587 (SEQ ID | 36.18 | Cyt | 95 | 96 |
| | | NO: 0001), U.S. Pat. No. 7,186,893 (SEQ | | | | |
| | | ID NO: 0001) | | | | |
| AGRMET1T218535 | Full-length | KR1019997002628 (SEQ ID | 30.96 | Cyt | 97 | 98 |
| | | NO: 0001), WO2007027776 | | | | |
| | | (SEQ ID NO: 0001), | | | | |
| | | WO2007027776 (SEQ ID | | | | |
| | | NO: 0007) | | | | |
| AGRMET1T581825 | Full-length | WO2005019414 (SEQ ID | 31.74 | Clostridium/ | 99 | 100 |
| | | NO: 0032, ABV98376, | | epsilon toxin | | |
| | | ABW00161 | | | | |
| AGRMET1T218655 | Full-length | WO2010099365 (SEQ ID | 56.79 | Clostridium/ | 101 | 102 |
| | | NO: 0029), WO2010099365 | | epsilon toxin | | |
| | | (SEQ ID NO: 0181), | | | | |
| | | WO2010099365 (SEQ ID | | | | |
| | | NO: 0182) | | | | |
| AGRMET1T594567 | Partial | AET40693, AY858558, | 23.50 | Clostridium/ | 103 | 104 |
| | | AB444205 | | epsilon toxin | | |
| AGRMET1T218847 | Full-length | BAG28156, ADJ41718, | 20.42 | Clostridium/ | 105 | 106 |
| | | ACZ07215 | | epsilon toxin | | |
| AGRMET1T655671 | Full-length | US20120066793 (SEQ ID | 95.27 | Clostridium/ | 107 | 108 |
| | | NO: 0009) | | epsilon toxin | | |
| AGRMET1T587003 | Full-length | WO2011014749 (SEQ ID | 44.57 | Clostridium/ | 109 | 110 |
| | | NO: 0008), US20110030096 | | epsilon toxin | | |
| | | (SEQ ID NO: 0008) | | | | |
| AGRMET1T218951 | Full-length | CP001748, CP002093, | 26.67 | Clostridium/ | 111 | 112 |
| | | CP001597 | | epsilon toxin | | |
| AGRMET1T218961 | Full-length | CP001748, CP002093, | 24.78 | Clostridium/ | 113 | 114 |
| | | CP001597 | | epsilon toxin | | |
| AGRMET1T619806 | Partial | WO2011014749 (SEQ ID | 38.61 | Clostridium/ | 115 | 116 |
| | | NO: 0009), US20110030096 | | epsilon toxin | | |
| | | (SEQ ID NO: 0009) | | | | |
| AGRMET1T616666 | Full-length | WO2011125015 (SEQ ID | 22.98 | Clostridium/ | 117 | 118 |
| | | NO: 9708), WO2011004263 | | epsilon toxin | | |
| | | (SEQ ID NO: 0032), | | | | |
| | | WO2011080595 (SEQ ID | | | | |
| | | NO: 0014) | | | | |
| AGRMET1T627497 | Full-length | U.S. Pat. No. 8,114,976 (SEQ ID | 21.23 | Clostridium/ | 119 | 120 |
| | | NO: 0740), WO2006044045 | | epsilon toxin | | |
| | | (SEQ ID NO: 0740) | | | | |
| AGRMET1T637067 | Full-length | ACA38725, AY858558, | 23.05 | Clostridium/ | 121 | 122 |
| | | AB444205 | | epsilon toxin | | |
| AGRMET1T143415 | Partial | WO2011014749 (SEQ ID | 28.16 | Clostridium/ | 123 | 124 |
| | | NO: 0026), US20110030096 | | epsilon toxin | | |
| | | (SEQ ID NO: 0026) | | | | |
| AGRMET1T218713 | Full-length | ABY46496 | 60.57 | Vip | 125 | 126 |
| AGRMET1T218461 | Full-length | ABY46496 | 49.73 | Vip | 127 | 128 |
| AGRMET1T218332 | Full-length | WO2010099365 (SEQ ID | 45.36 | Vip | 129 | 130 |
| | | NO: 0024), WO2010099365 | | | | |
| | | (SEQ ID NO: 0121), | | | | |
| | | WO2010099365 (SEQ ID | | | | |
| | | NO: 0122) | | | | |
| AGRMET1T218708 | Partial | WO2010099365 (SEQ ID | 48.21 | Vip | 131 | 132 |
| | | NO: 0024, WO2010099365 | | | | |
| | | (SEQ ID NO: 0121, | | | | |
| | | WO2010099365 (SEQ ID | | | | |
| | | NO: 0122 | | | | |
| AGRMET1T219045 | Full-length | WO2010099365 (SEQ ID | 42.01 | Vip | 133 | 134 |
| | | NO: 0024), WO2010099365 | | | | |
| | | (SEQ ID NO: 0121), | | | | |
| | | WO2010099365 (SEQ ID | | | | |
| | | NO: 0122) | | | | |
| AGRMET1T218600 | Full-length | US20120121607 (SEQ ID | 46.27 | Vip | 135 | 136 |
| | | NO: 0006), BAK40944, | | | | |
| | | AB604032 | | | | |
| AGRMET1T219032 | Full-length | JP2011526150 (SEQ ID | 41.21 | Vip | 137 | 138 |
| | | NO: 0041), JP2011526150 | | | | |
| | | (SEQ ID NO: 0147), | | | | |
| | | JP2011526150 (SEQ ID | | | | |
| | | NO: 0148) | | | | |
| AGRMET1T218883 | Full-length | AEB20803, JP2011526150 | 29.53 | Vip | 139 | 140 |
| | | (SEQ ID NO: 0041), | | | | |
| | | JP2011526150 (SEQ ID | | | | |
| | | NO: 0147) | | | | |
| AGRMET1T219117 | Full-length | HQ639674, AEC11570, | 33.23 | Vip | 141 | 142 |
| | | HQ639679 | | | | |
| AGRMET1T697885 | Partial | JP2011526150 (SEQ ID | 56.76 | Cry | 143 | 144 |
| | | NO: 0065), JP2011526150 | | | | |
| | | (SEQ ID NO: 0195), | | | | |
| | | JP2011526150 (SEQ ID | | | | |
| | | NO: 0196) | | | | |
| AGRMET1T218491 | Full-length | WO2010099365 (SEQ ID | 67.48 | Cry | 145 | 146 |
| | | NO: 0023), WO2010099365 | | | | |
| | | (SEQ ID NO: 0109), | | | | |
| | | WO2010099365 (SEQ ID | | | | |
| | | NO: 0110) | | | | |
| AGRMET1T218662 | Full-length | WO2010099365 (SEQ ID | 59.95 | Cry | 147 | 148 |
| | | NO: 0023), WO2010099365 | | | | |
| | | (SEQ ID NO: 0109), | | | | |
| | | WO2010099365 (SEQ ID | | | | |
| | | NO: 0110) | | | | |
| AGRMET1T218366 | Partial | HQ221867, ADO51070 | 89.64 | Cry | 149 | 150 |
| AGRMET1T218673 | Full-length | WO2011014749 (SEQ ID | 29.94 | Cry | 151 | 152 |
| | | NO: 0016), US20110030096 | | | | |
| | | (SEQ ID NO: 0016) | | | | |
| AGRMET1T218582 | Partial | JP2011526150 (SEQ ID | 34.62 | Cry | 153 | 154 |
| | | NO: 0009), JP2011526150 | | | | |
| | | (SEQ ID NO: 0083), | | | | |
| | | JP2011526150 (SEQ ID | | | | |
| | | NO: 0084) | | | | |
| AGRMET1T218580 | Partial | JP2011526150 (SEQ ID | 31.44 | Cry | 155 | 156 |
| | | NO: 0009), JP2011526150 | | | | |
| | | (SEQ ID NO: 0083), | | | | |
| | | JP2011526150 (SEQ ID | | | | |
| | | NO: 0084) | | | | |
| AGRMET1T697805 | Partial | ADO51070, HQ221867 | 89.19 | Cry | 157 | 158 |
| AGRMET1T697793 | Partial | ABY46520 | 80.85 | Cry | 159 | 160 |
| AGRMET1T697907 | Partial | BAC77648, AB112346 | 74.68 | Cry | 161 | 162 |
| AGRMET1T166424 | Partial | CAA09344, CAD30080, | 100.00 | Cry | 163 | 164 |
| | | AJ010753 | | | | |
| AGRMET1T697865 | Partial | US Pat. U.S. Pat. No. 4,652,628 (SEQ ID | 100.00 | Cry | 165 | 166 |
| | | NO: 0001) | | | | |
| AGRMET1T697862 | Partial | US Appl. US20100017914 | 98.00 | Cry | 167 | 168 |
| | | (SEQ ID NO: 0078) | | | | |
| AGRMET1T218478 | Partial | WO2010099365 (SEQ ID | 36.63 | Cry | 169 | 170 |
| | | NO: 0041), WO2010099365 | | | | |
| | | (SEQ ID NO: 0183), | | | | |
| | | WO2010099365 (SEQ ID | | | | |
| | | NO: 0184) | | | | |
| AGRMET1T697789 | Partial | WO2010099365 (SEQ ID | 68.84 | Cry | 171 | 172 |
| | | NO: 0023) | | | | |
| AGRMET1T697804 | Partial | ACF15199, CN101824419 | 61.86 | Cry | 173 | 174 |
| | | (SEQ ID NO: 0002), | | | | |
| | | CN101824419 (SEQ ID | | | | |
| | | NO: 0001) | | | | |
| AGRMET1T697798 | Partial | US Pat. U.S. Pat. No. 5,424,410 (SEQ ID | 79.25 | Cry | 175 | 176 |
| | | NO: 0029) | | | | |
| AGRMET1T218291 | Full-length | WO2010099365 (SEQ ID | 31.65 | Cry | 177 | 178 |
| | | NO: 0041), WO2010099365 | | | | |
| | | (SEQ ID NO: 0183), | | | | |
| | | WO2010099365 (SEQ ID | | | | |
| | | NO: 0184) | | | | |
| AGRMET1T218530 | Partial | JP2011526150 (SEQ ID | 61.96 | Cry | 179 | 180 |
| | | NO: 0193), JP2011526150 | | | | |
| | | (SEQ ID NO: 0194), | | | | |
| | | WO2009158470 (SEQ ID | | | | |
| | | NO: 0264) | | | | |
| AGRMET1T697889 | Partial | EP1947184 (SEQ ID | 65.07 | Cry | 181 | 182 |
| | | NO: 0024), US20040210964 | | | | |
| | | (SEQ ID NO: 0006) | | | | |
| AGRMET1T218952 | Partial | AZE84646, WO2011014749 | 48.39 | Cry | 183 | 184 |
| | | (SEQ ID NO: 0016) | | | | |
| AGRMET1T218383 | Partial | ADO51070, HQ221867 | 85.04 | Cry | 185 | 186 |
| AGRMET1T218678 | Partial | JP2011526150 (SEQ ID | 44.29 | Cry | 187 | 188 |
| | | NO: 0193), JP2011526150 | | | | |
| | | (SEQ ID NO: 0194), | | | | |
| | | WO2009158470 (SEQ ID | | | | |
| | | NO: 0264) | | | | |
| AGRMET1T218616 | Partial | JP2011526150 (SEQ ID | 48.53 | Cry | 189 | 190 |
| | | NO: 0069), WO2009158470 | | | | |
| | | (SEQ ID NO: 0131) | | | | |
| AGRMET1T218876 | Full-length | WO2010099365 (SEQ ID | 26.41 | Cry | 191 | 192 |
| | | NO: 0040), WO2010099365 | | | | |
| | | (SEQ ID NO: 0117), | | | | |
| | | WO2010099365 (SEQ ID | | | | |
| | | NO: 0118) | | | | |
| AGRMET1T218404 | Partial | AZE84673, WO2011014749 | 89.33 | Cry | 193 | 194 |
| | | (SEQ ID NO: 0043) | | | | |
| AGRMET1T218319 | Full-length | WO2010099365 (SEQ ID | 40.03 | Cry | 195 | 196 |
| | | NO: 0035), WO2010099365 | | | | |
| | | (SEQ ID NO: 0159), | | | | |
| | | WO2010099365 (SEQ ID | | | | |
| | | NO: 0160) | | | | |
| AGRMET1T219034 | Full-length | WO2010099365 (SEQ ID | 23.01 | Cry | 197 | 198 |
| | | NO: 0040), WO2010099365 | | | | |
| | | (SEQ ID NO: 0117), | | | | |
| | | WO2010099365 (SEQ ID | | | | |
| | | NO: 0118) | | | | |
| AGRMET1T697771 | Full-length | WO2010099365 (SEQ ID | 29.08 | Cry | 199 | 200 |
| | | NO: 0041), WO2010099365 | | | | |
| | | (SEQ ID NO: 0183), | | | | |
| | | WO2010099365 (SEQ ID | | | | |
| | | NO: 0184) | | | | |
| AGRMET1T219087 | Partial | WO2010099365 (SEQ ID | 30.50 | Cry | 201 | 202 |
| | | NO: 0042), WO2010099365 | | | | |
| | | (SEQ ID NO: 0185), | | | | |
| | | WO2010099365 (SEQ ID | | | | |
| | | NO: 0186) | | | | |
| AGRMET1T218636 | Full-length | WO2010099365 (SEQ ID | 26.65 | Cry | 203 | 204 |
| | | NO: 0042), WO2010099365 | | | | |
| | | (SEQ ID NO: 0185), | | | | |
| | | WO2010099365 (SEQ ID | | | | |
| | | NO: 0186) | | | | |
| AGRMET1T697780 | Partial | WO2011014749 (SEQ ID | 32.04 | Cry | 205 | 206 |
| | | NO: 0016), US20110030096 | | | | |
| | | (SEQ ID NO: 0016) | | | | |

### Example 5

### Construction of Synthetic Toxin Genes

In some experiments, synthetic toxin sequences are generated. These synthetic sequences may have an altered DNA sequence relative to the parent toxin sequence, and encode a protein that is collinear with the parent toxin protein to which it corresponds, but optionally lacks the C-terminal “crystal domain” present in many delta-endotoxin proteins.

In some other experiments, modified versions of synthetic genes are designed such that the resulting peptide is targeted to a plant organelle, such as the endoplasmic reticulum or the apoplast. Peptide sequences known to result in targeting of fusion proteins to plant organelles are known in the art. For example, the N-terminal region of the acid phosphatase gene from the White Lupin Lupinus albus (Miller et al., Plant Physiology 127: 594-606, 2001) is known to result in endoplasmic reticulum targeting of heterologous proteins. If the resulting fusion protein also contains an endoplasmic retention sequence comprising the peptide N-terminus-lysine-aspartic acid-glutamic acid-leucine (i.e. the “KDEL” motif) at the C-terminus, the fusion protein can be targeted to the endoplasmic reticulum. If the fusion protein lacks an endoplasmic reticulum targeting sequence at the C-terminus, the protein can be targeted to the endoplasmic reticulum, but can ultimately be sequestered in the apoplast.

### Example 6

### Expression in Bacillus Spp. Cell and Pseudomonas Spp. Cell

In some experiments, biotoxins having sequences as disclosed herein are synthesized and cloned into a vector suitable for Bacillus spp. or Pseudomonas spp., by using known cloning methods. For transformation, Bacillus spp. or Pseudomonas spp. cultures are prepared appropriately according to transformation procedures known in the art. The resulting Bacillus spp. or Pseudomonas spp. recombinant strains, containing the vector with the toxin genes are cultured individually on a conventional growth media, such as CYS media (10 g/l Bacto-casitone; 3 g/l yeast extract; 6 g/l KH₂PO₄; 14 g/l K₂HPO₄; 0.5 mM MgSO₄; 0.05 mM MnCl₂; 0.05 mM FeSO₄), until sporulation is evident by microscopic examination. Samples are prepared and tested for activity in bioassays.

### Example 7

### Functional In Vitro Bioassays

DNA molecules encoding toxins or predicted toxin domains as disclosed in the present application are separately cloned into a suitable E. coli expression vector containing a selectable antibiotic resistant marker, followed by transformation of E. coli competent cells with individual plasmids. For each construct, a single colony is inoculated in LB medium supplemented with the antibiotic and grown overnight at 37° C. The following day, fresh media are inoculated with 1% of overnight culture and grown at 37° C. to logarithmic phase. Each cell pellet is suspended in a Tris buffer (20 mM Tris-Cl buffer, pH 7.4, 200 mM NaCl, 1 mM DTT) with protease inhibitors and sonicated. Expression of the toxin proteins are confirmed by SDS-PAGE analysis. Toxin proteins are then purified by techniques known in the art (see, e.g., Sambrook and Russell, 2001, supra). Purified proteins are tested in insect assays with appropriate controls. A 5 day read of the plates show that the toxin proteins have pesticidal activity against Diamondback moth and Southwestern corn borer pests.

### Example 8

### Additional Assays for Pesticidal Activity

The ability of a pesticidal protein to act as a pesticide upon a pest is often assessed in a number of ways. One way well known in the art is to perform a feeding assay. In such a feeding assay, one exposes the pest to a sample containing either toxins/compounds to be tested, or control samples. Often this is performed by placing the material to be tested, or a suitable dilution of such material, onto a material that the pest will ingest, such as an artificial diet. The material to be tested may be composed of a liquid, solid, or slurry. The material to be tested may be placed upon the surface and then allowed to dry. Alternatively, the material to be tested may be mixed with a molten artificial diet, and then dispensed into the assay chamber. The assay chamber may be, for example, a cup, a dish, or a well of a microtiter plate.

Assays for sucking pests (for example aphids) may involve separating the test material from the insect by a partition, ideally a portion that can be pierced by the sucking mouth parts of the sucking insect, to allow ingestion of the test material. Often the test material is mixed with a feeding stimulant, such as sucrose, to promote ingestion of the test compound.

Other types of assays can include microinjection of the test material into the mouth, or gut of the pest, as well as development of transgenic plants, followed by test of the ability of the pest to feed upon the transgenic plant. Plant testing may involve isolation of the plant parts normally consumed, for example, small cages attached to a leaf, or isolation of entire plants in cages containing insects. Other methods and approaches to assay pests are known in the art, and can be found, for example in Robertson and Preisler (Pesticide Bioassays with Arthropods, CRC Press, Science, 1992).

### Example 9

### Transformation of Plants, Plant Cells, and Tissues

Vector construction: Each of the coding regions of the genes of the invention is connected independently with appropriate promoter and terminator sequences for expression in plants. Such sequences are well known in the art and may include a viral CaMV 35S promoter, a rice actin promoter or a maize ubiquitin promoter for expression in monocots, the Arabidopsis UBQ3 promoter or for expression in dicots, and the NOS or OCS terminators. Techniques for producing and confirming promoter-gene-terminator constructs also are well known in the art. The following examples are offered by way of illustrations and not by way of limitation.

### Production of the Novel Biotoxin Proteins in Transformed Plants

Expression cassettes that include either full-length or truncated forms of the biotoxin proteins as described above are made in suitable shuttle vectors by routine procedures, using a CaMV 35S promoter (Howell and Hull, Virology 1978) and a ubiquitin promoter (Christensen et al., Plant Mol. Biol. 1992). In some instances, to optimize expression efficiency of the biotoxin proteins in the host plant, the codon usage of the open reading frame is adapted to that of the host plant such that alternative codons are used while encoding for the same protein. Such altered sequences are generated by the Reverse Translate software, which is a codon-optimization software that can be found on the World Wide Web at bioinformatics.org/sms2/rev_trans.html. Plant cells, including e.g. barley, wheat, triticale, corn, cotton, and rice cells, are then transformed with the resulting recombinant vectors.

Barley, wheat, triticale, corn cells are stably transformed by either Agrobacterium-mediated transformation or by electroporation using wounded and enzyme-degraded embryogenic callus, as described in, e.g., Henzel et al. (Inter. J. of Plant Genomics, 2009); PCT Appl. No. WO 92/09696 and U.S. Pat. No. 5,641,664.

Cotton cells are stably transformed by Agrobacterium-mediated transformation as described by, e.g., Umbeck et al., 1987, and U.S. Pat. No. 5,004,863.

Rice cells are stably transformed by essentially following the method described by Hiei et al., Plant J. August, 6(2):271-82, 1994; and PCT Appl. No. WO 92/09696.

Regenerated transformed corn, cotton and rice plants are selected by Northern blot, Southern blot, ELISA, and insecticidal effect, or a combination of these techniques. Biotoxin sequence-containing progeny plants show improved resistance to insects compared to untransformed control plants with appropriate segregation of insect resistance and the transformed phenotype. Protein and RNA measurements show that increased insect resistance is linked with higher expression of the novel Cry protein in the plants.

Agrobacterium-Mediated Transformation of Maize Cell with the Toxin-Encoded Sequences of the Invention

Maize embryos are isolated from the 8-12 DAP ears, and those embryos of 0.8-1.5 mm in size are used for transformation. Embryos are plated with the scutellum side up on a suitable incubation media, and optionally incubated overnight at 25° C. in the dark. Embryos are then contacted with an Agrobacterium strain containing the appropriate vectors for Ti plasmid mediated transfer for 5-10 min, and then plated onto co-cultivation media for 3 days (25° C. in the dark). After co-cultivation, explants are transferred to recovery period media for five days (at 25° C. in the dark). Explants are incubated in suitable selection media for up to eight weeks, depending on the nature and characteristics of the particular selection utilized. After the selection period, the resulting callus is transferred to embryo maturation media, until the formation of mature somatic embryos is observed. The resulting mature somatic embryos are then placed under low light, and the process of regeneration is initiated as known in the art. The resulting shoots are allowed to root on rooting media, and the resulting plants are transferred to nursery pots and propagated as transgenic plants.

Transformation of Maize Cells with the Toxin-Encoded Sequences of the Invention by Using Aerosol Beam Technology.

Maize embryos are isolated from the 8-12 DAP ears, and those embryos of 0.8-1.5 mm in size are used for transformation. Embryos are plated scutellum side-up on a suitable incubation media, such as DN62A5S media (3.98 g/L N6 Salts; 1 mL/L of 1000× Stock N6 Vitamins; 800 mg/L L-Asparagine; 100 mg/L Myo-inositol; 1.4 g/L L-Proline; 100 mg/L Casaminoacids; 50 g/L sucrose; 1 mL/L of 1 mg/mL Stock 2,4-D), and incubated overnight at 25° C. in the dark. The resulting explants are transferred to mesh squares (30-40 per plate), then transferred onto osmotic media for 30-45 minutes, and subsequently transferred to a beaming plate (see, for example, PCT Appl. No. WO200138514 and U.S. Pat. No. 5,240,842).

DNA constructs designed to express the sequences of the invention in plant cells are accelerated into plant tissue using an aerosol beam accelerator, using conditions essentially as described in PCT Appl. No. WO200138514. After beaming, embryos are incubated for 30 min on osmotic media, and then placed onto incubation media overnight at 25° C. in the dark. To avoid damaging beamed explants, they are incubated for at least 24 hours prior to transfer to recovery media. Embryos are then spread onto recovery period media, for 5 days, 25° C. in the dark, transferred to a selection media. Explants are incubated in selection media for up to eight weeks, depending on the nature and characteristics of the particular selection utilized. After the selection period, the resulting callus is transferred to embryo maturation media, until the formation of mature somatic embryos is observed. The resulting mature somatic embryos are then placed under low light, and the process of regeneration is initiated by methods known in the art. The resulting shoots are allowed to root on rooting media, and the resulting plants are transferred to nursery pots and propagated as transgenic plants.

A number of embodiments of the invention have been described. Nevertheless, it will be understood that elements of the embodiments described herein can be combined to make additional embodiments and various modifications may be made without departing from the spirit and scope of the invention. Accordingly, other embodiments, alternatives and equivalents are within the scope of the invention as described and claimed herein.

Headings within the application are solely for the convenience of the reader, and do not limit in any way the scope of the invention or its embodiments.

All publications and patent applications mentioned in this specification are herein incorporated by reference to the same extent as if each individual publication or patent application was specifically can individually indicated to be incorporated by reference.

What is claimed is:

1. A method for identifying a nucleic acid sequence encoding a biotoxin, said method comprising:
a) generating a mixed population of extrachromosomal DNA molecules from a plurality of microbial isolates;
b) establishing a metagenomic sequence dataset comprising nucleic acid sequences derived from said mixed population of extrachromosomal DNA molecules;
c) processing sequence data of said metagenomic sequence dataset to define at least one nucleic acid sequence contig; and
d) identifying a nucleic acid sequence that encodes a biotoxin by comparing said at least one nucleic acid sequence contig from step (c) with known biotoxin sequences.

2. A method according to claim 1, said method further comprising a step of determining the taxonomic classification of said microbial isolates.

3. A method according to claim 1, wherein said plurality of microbial isolates are pre-selected for the ability to produce at least one biotoxin.

4. A method according to claim 1, said method further comprising a step of determining whether said nucleic acid sequence identified from step (d) encodes a novel biotoxin, wherein the nucleic acid sequence of said novel toxin identified shares less than 30% sequence identity with any known biotoxin sequence.

5. A method of claim 1, wherein said plurality of microbial isolates comprises at least 12 microbial isolates.

6. A method according to claim 1, wherein at least one of said microbial isolates is a bacterium.

7. A method according to claim 6, wherein said bacterium is of a genus selected from the group consisting of Bacillus, Brevibacillus, Clostridia, Paenibacillus, Photorhabdus, Pseudomonas, Serratia, Streptomyces, and Xenorhabdus.

8. A method according to claim 1, wherein said metagenomic sequence dataset is generated by a direct sequencing procedure that excludes molecular cloning.

9. An isolated nucleic acid molecule comprising a nucleic acid sequence identified by a method according to any one of claims 1-8.

10. An isolated nucleic acid molecule comprising:
(a) a nucleic acid sequence hybridizing under high stringency conditions to any one of the nucleotide sequences in the Sequence Listing, a complement thereof or a fragment of either; or
(b) a nucleic acid sequence exhibiting 70% or greater sequence identity to any one of the nucleotide sequences in the Sequence Listing, a complement thereof or a fragment of either; or
(c) a nucleic acid sequence encoding an amino acid sequence exhibiting 50% or greater sequence identity to any one of the amino acid sequences in the Sequence Listing.

11. A nucleic acid construct comprising a nucleic acid molecule according to claim 10, wherein said nucleic acid molecule is operably linked to a heterologous nucleic acid.

12. A host cell comprising a nucleic acid construct according to claim 11.

13. A host cell according to claim 12, wherein said host cell is a plant cell or a microbial cell.

14. A host organism comprising a host cell according to claim 12.

15. A biological sample or progeny derived from a host organism according to claim 14.

16. A method for conferring pesticidal activity to an organism, said method comprising introducing into said organism a nucleic acid molecule according to claim 10, wherein said nucleic acid molecule is transcribed and results in an elevated resistance of said organism to a pest as compared to a control organism.

17. An isolated polypeptide, wherein said polypeptide is encoded by a nucleic acid molecule comprising:
(a) a nucleic acid sequence hybridizing under high stringency conditions to any one of the nucleotide sequences in the Sequence Listing, a complement thereof or a fragment of either; or
(b) a nucleic acid sequence exhibiting 70% or greater sequence identity to any one of the nucleotide sequences in the Sequence Listing, a complement thereof or a fragment of either; or
(c) a nucleic acid sequence encoding an amino acid sequence exhibiting 50% or greater sequence identity to any one of the amino acid sequences in the Sequence Listing.

18. A polypeptide according to claim 17, wherein said polypeptide has pesticidal activity.

19. A composition comprising a polypeptide according to claim 18.

20. A method for controlling a pest, said method comprising contacting or feeding said pest with a pesticidally-effective amount of a polypeptide according to claim 18.
